(19) United States
(12) Patent Application Publication
(10) Pub. No.: US 2023/0119920 A1
(43) Pub. Date: Apr. 20, 2023

(54) METHOD FOR PRODUCING REDUCED GLYCOL FATTY ALCOHOL ETHOXYLATES, REDUCED GLYCOL SULFATE ETHOXYLATED SURFACTANTS, AND PRODUCTS

(71) Applicant: The Procter & Gamble Company, Cincinnati, OH (US)

(72) Inventor: EDWARD DEWEY SMITH, III, MASON, OH (US)

(21) Appl. No.: 18/076,752

(22) Filed: Dec. 7, 2022

Related U.S. Application Data
(63) Continuation of application No. PCT/US2021/036571, filed on Jun. 9, 2021.
(60) Provisional application No. 63/042,326, filed on Jun. 22, 2020.
(60) Provisional application No. 63/042,337, filed on Jun. 22, 2020.
(60) Provisional application No. 63/042,347, filed on Jun. 22, 2020.

(51) Int. Cl. C11D 1/72; C11D 1/29; C11D 11/00; A61Q 19/10; A61K 8/39; A61K 8/46

(52) U.S. Cl. CPC C11D 1/72; A61K 8/39; A61K 8/463; A61Q 19/10; C11D 1/29; C11D 11/0017; C11D 11/0023

(57) ABSTRACT

A method of producing a reduced glycol fatty alcohol ethoxylate material may include removing at least a portion of glycol in the fatty alcohol ethoxylate material. A method of producing a reduced glycol sulfate ethoxylated sulfate surfactant may include, removing at least a portion of glycol in an ethoxylated fatty alcohol raw material before the ethoxylated fatty alcohol raw material is sulfated to produce the reduced glycol sulfate ethoxylated sulfate surfactant. A surfactant including an ethoxylated fatty alcohol with 100 ppm or less by weight of free glycol. A surfactant including a sulfated ethoxylated alcohol with 100 ppm or less by weight of diethylene glycol monosulfate sodium.

### FIELD OF THE INVENTION

This application is directed to methods of producing a reduced glycol fatty alcohol ethoxylate, methods of producing reduced glycol sulfate ethoxylated surfactants, surfactants with 100 ppm or less by weight of free glycol, diethylene glycol monosulfate sodium, and products containing the same.

### BACKGROUND OF THE INVENTION

Fatty alcohol ethoxylates are used are in many industries. For example, they can be used as non-ionic surfactants in detergents and cleansers. They can also be an intermediate in the production of other surfactants through processes like sulfation. Current processes used to produce fatty alcohol ethoxylates, can result in the inclusion of unwanted byproducts which either remain as part of the fatty alcohol ethoxylate material as it is sold or used, or are further processed along with the fatty alcohol ethoxylate into a different surfactant which can result in additional or different unwanted byproducts. These byproducts can be problematic for users of the fatty alcohol ethoxylate raw material itself or of the materials produced from the fatty alcohol ethoxylate material. As such, there is a need for a fatty alcohol material with a better byproduct profile.

### SUMMARY OF THE INVENTION

One exemplary method includes producing a reduced glycol fatty alcohol ethoxylate material comprising, removing at least a portion of glycol in the fatty alcohol ethoxylate material after formation of the fatty alcohol ethoxylate material.

Another exemplary method includes reducing the amount of ethylene glycol oligomer in an alcohol ethoxylate material, comprising extracting ethylene glycol oligomer from the alcohol ethoxylate material.

These and other methods can be found below in the Detailed Description section.

### BRIEF DESCRIPTION OF THE DRAWINGS

FIG. 1 is a graph showing the dioxane formed (ppm) over time (weeks) in raw materials aged at 30° C.;

FIG. 2 is a graph showing the dioxane formed (ppm) over time (weeks) in raw materials aged at 40° C.;

FIG. 3 is a graph showing dioxane formation rate in ppm/ week versus pH for a 15% active SLE1S composition at 50° C.;

FIG. 4 is a graph showing pH impact on dioxane levels in a 45% active ST2S raw material after 20 days of aging at 65° C.

FIG. 5 is a graph showing the dioxane formed (ppm) over time (weeks) in raw materials aged at 50° C.;

FIG. 6 is a graph of the dioxane formed (ppm) in a personal cleansing composition (body wash) made with scrubbed sodium trideceth-2 sulfate at varying temperatures over time (weeks);

FIG. 7 is a graph showing the amount of dioxane formed (ppm) over time (weeks) in three scrubbed 45% active sodium trideceth-2 sulfate surfactant raw material samples;

FIG. 8 is a graph showing the Peak Area Concentration Normalized to Initial Concentration of polyethylene glycol (n) monosulfate in samples of sodium laureth-1 sulfate (SLE1S), sodium laureth-3 sulfate (SLE3 S), and sodium trideceth-2 sulfate (ST2S), versus the stability sample temperature;

FIG. 9 is a graph of the temporal dioxane growth in raw material samples plotting dioxane growth in the sample, difference from the initial (ppm) versus the LCMS counts at 2 months (difference from the ambient sample); and

FIG. 10 is a graph of glycol primary byproducts found in a sample of 2-tridecyl alcohol ethoxylate.

### DETAILED DESCRIPTION OF THE INVENTION

Fatty alcohol ethoxylates are a class of compounds used in many different industries. One of the more common uses of this class of compounds is that of a surfactant. As a surfactant, the properties of the compounds in this class can vary widely given that these compounds can be made from a wide range of materials. Simply put, fatty alcohol ethoxylates are commonly produced by the reaction of a fatty alcohol with ethylene oxide. The reaction generally occurs with a basic catalyst such as an alkali metal hydroxide, allowing ethoxylate units to sequentially add to the fatty alcohol, resulting in a relatively broad distribution of fatty alcohol ethoxylates, FAE(n), where n has an average value from about 1 to about 20; and also has a distribution of ethoxylates for any value of n. The value of n is generally expressed on a molar basis as the ratio of EO moles added to fatty alcohol moles added to the reaction.

Specific catalysts can also be used to tailor the distribution of ethoxylates for any value of n, for example, to make it narrower, which can be used to alter the properties. There is a large class of fatty alcohols suitable for ethoxylation and each can contribute to a fatty alcohol ethoxylate with a different set of properties. The same can be said for the ethylene oxide component, where the chain length of this component can impact the properties of the final fatty alcohol ethoxylate. Some examples of fatty alcohol ethoxylates include 2-tridecyl alcohol ethoxylate (TDA-2), alcohol ethoxylate-2 (AE-2), 3-tridecyl alcohol (TDA-3), alcohol ethoxylate-1 (AE-1), alcohol ethoxylate 1.8 (AE-1.8), and combinations thereof.

The reaction of the fatty alcohol and the ethylene oxide can also produce byproducts, i.e. compounds that are not a fatty alcohol ethoxylate. One class of materials that can be a byproduct of this reaction is free glycols, particularly an ethylene glycol oligomer. Free glycols are ethylene oxide addition products, i.e., oligomers of ethylene oxide that are not attached to a fatty alcohol. Generally, FAE can contain about 0.2 wt% to about 1.5 wt% free glycols, or about 0.5 wt% to about 1 wt% free glycols. These free glycols may interfere with the ability to formulate with the alcohol ethoxylates particularly where there is a desire to limit those types of byproduct materials in a final product.

There is another issue, however. Fatty alcohol ethoxylates are sometimes further reacted to produce other compounds, like alcohol ethoxylated sulfate surfactants (AES), by reacting the fatty alcohol ethoxylates with gas phase sulfur trioxide (SO₃) in a falling film reactor (FFR) or chlorosulfonic acid (HSO₃Cl) in a batch process, closely followed by a neutralization step to produce an aqueous surfactant paste or solution having a prescribed pH, between about pH 5 to about pH 13. The surfactants generally are neutralized to an activity, which is a surfactant concentration by weight, between about 25% and about 70%. These ethoxylated sulfate surfactants can include, for example, sodium laureth(n) sulfate (SLEnS) and sodium trideceth(n) sulfate (STnS), where n is generally from 0.5 to 4. Exemplary SLEnS surfactants can include sodium laureth-1 sulfate, sodium laureth-1.8 sulfate, sodium laureth-2 sulfate, sodium laureth-2.5 sulfate, and sodium laureth-3 sulfate. Exemplary STnS surfactants can include sodium trideceth-2 sulfate and sodium trideceth-3 sulfate.

During the sulfation, primary byproducts, like free glycols, also become sulfated to form secondary byproducts, glycol monosulfates and disulfates. In addition, under conditions of abundant SO₃, scission of EO oligomers, whether on the free glycols, the FAE, or the sulfated FAE, also produces secondary byproducts such as the aforementioned glycol sulfates and disulfates, and longer glycol mono and disulfates in general. Occasionally, cyclization of certain by-products can occur to form secondary byproducts like 1,4-dioxane. Recently, there has been a desire to limit the amount of 1,4-dioxane.

1,4-dioxane is a known secondary byproduct of the combination of ethoxylation and sulfation processes to form surfactants (see, Norman, Sulfonation and Sulfation Processes, The Chemithon Corporation, 1997.) Although it can be present at a very low level in a surfactant or a cleansing product containing a surfactant, often below 50 ppm, there is now a desire to further reduce and/or eliminate it from a surfactant and/ or surfactant containing products. One process to remove 1,4-dioxane from a surfactant is to scrub the 1,4-dioxane from the ethoxylated sulfate surfactant raw material using a dioxane removal system. Some exemplary methods of scrubbing dioxane can be found, for example, in U.S. App. No. 17/184,634 and U.S. Pat. No. 4,285,881. Dioxane scrubbing utilizes specialized equipment which can be costly, energy-intensive, and utilizes additional processing time, all of which lead to a higher manufacturing cost to the maker of the surfactant and surfactant containing products.

Research into the 1,4-dioxane levels in products has resulted in some learnings. First, it is understood that driving the sulfation reaction in the manufacturing of an ethoxylated sulfate surfactant by using greater than 1 mole of sulfate source to the feedstock fatty alcohol, results in a dramatic increase in the production of 1,4-dioxane (see, Norman, Sulfonation and Sulfation Processes, The Chemithon Corporation, 1997). In order to prevent the creation of additional 1,4-dioxane, many surfactant raw material manufacturers will not completely sulfate the feedstock FAE to 100% completion, for example they may perform this reaction at less than about a 1.03:1 mole ratio, and run the reaction under a set of conditions that result in completion of the sulfation to only about 96% or 97% or 98%.

Second, as ethoxylated sulfate surfactant raw material is transported at an elevated temperature or sits in a heated tank waiting to be incorporated into a product at a manufacturing plant, the level of dioxane can increase, sometimes substantially. While the reason for this wasn’t understood, the result needed to be mitigated. So, to mitigate the phenomenon, such raw materials were assigned a shelf life and were used as soon as possible in the manufacturing plant. This alone, however, is insufficient to meet the current desired levels of dioxane in a finished product.

As part of an investigation into a potential solution to meet the desired dioxane levels, pH of a surfactant raw material is studied. An SLE1S 70% active surfactant paste is obtained commercially having a pH of 12.97, and an initial dioxane concentration of 5.76 ppm (8.2 ppm 100% active). Part of the paste is sealed in containers, the remaining paste is neutralized using citric acid to pH 7.8 and placed in sealed containers. Containers are stored at 50° C. or 65° C., and dioxane measured periodically. All pH values are obtained using a 10% dilution of raw material with 90% water, at 21° C. As can be seen in Table 1, dioxane growth from the initial time point is substantially lower at neutral pH for the 70% active surfactant paste. The numbers in parenthesis are calculated at a 100% active basis.

TABLE 1

| Sample | Storage Temperature | Initial | 1 wk | 2wks | 3 wks | 4 wks | 8 wks |
|---|---|---|---|---|---|---|---|
| SLE1S pH 7.8 | 50° C. | 0 (reference) | | 0.94 (1.34) | | 2.07 (2.96) | 2.58 (3.69) |
| | 65° C. | 0 (reference) | 5.35 (7.64) | | | | |
| SLE1S pH 12.97 | 50° C. | 0 (reference) | | 3.82 (5.46) | | 6.74 (9.63) | 8.56 (12.2) |
| | 65° C. | 0 (reference) | 12.74 (18.2) | | | | |

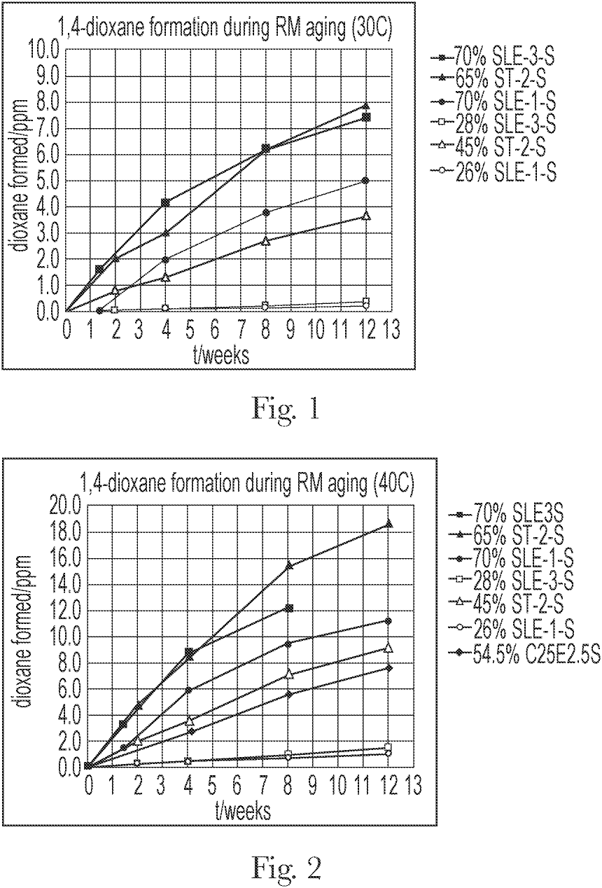

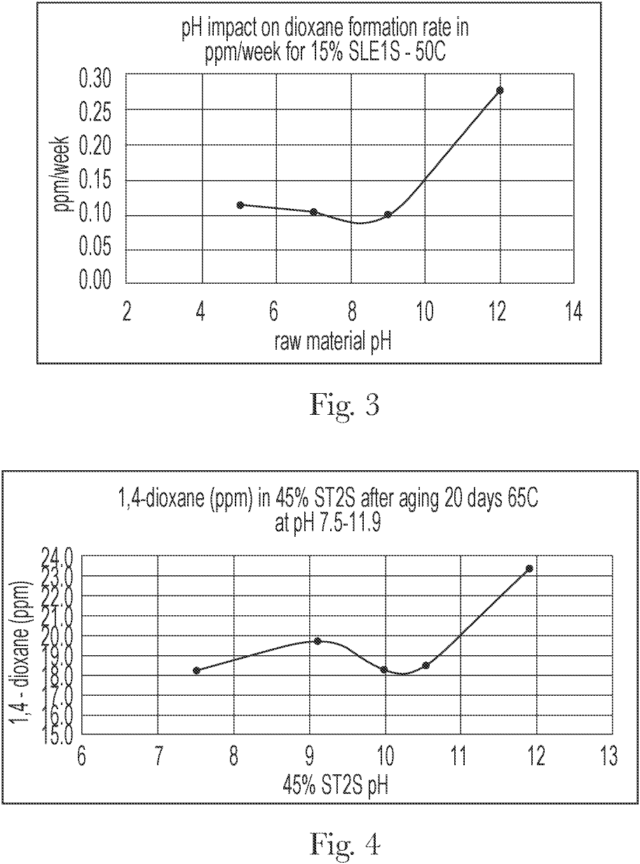

Thus, it appears AES raw materials at pH ≥ about 12 will rather quickly produce 1,4-dioxane when stored at typical temperatures (like 30-40° C.). This seems especially true if the surfactant activity is ≥ 45%, see FIGS. 1 and 2. Though, the same trend can be found, albeit to a lesser extent at lower concentrations. For example, a 15% active SLE1S paste is subjected to aging at 50° C. The results, in FIG. 3, show a relationship between the pH and the amount of dioxane growth over time, specifically, beginning at a pH of about 9, there is a steep increase in the formation of dioxane. Also, the issue is more apparent at higher temperatures (see FIGS. 1 and 2).

We have found that when the pH of these surfactants is controlled to be between about 7 to about 8 (as 10% solution) by means of adding an organic or inorganic buffer the growth rate of 1,4-dioxane is slowed down significantly (see Table 1). This helps to ensure a surfactant paste can be stored, handled, and transported for a much longer period of time. For example, it allows for an increase in the shelf life of these materials from less than 4 weeks to greater than 2 months, which then in turn allows more flexibility in the transportation and storage of these surfactant raw materials.

While a neutral pH of about 7 to about 8 has its advantages, one of the main reasons the pH of surfactant pastes is currently maintained at a pH of about 12 or above is that it is self-preserving, thus no separate preservative is needed. Looking at the pH curves, see FIGS. 3 and 4, there is a shift in the higher active percentage ST2S such that the dioxane growth doesn’t continue increasing at a continuous rate until above pH 10.5 and is still similar to the level seen at a pH of 9 or pH 11. This allows some room to bring down the pH to reduce the rate of growth of dioxane in the raw material, but also utilize a pH which still has some micro hostility, like a pH of 10-11.

Examples of buffers for use in the neutralization or reduction of pH of a surfactant raw material are organic buffers. Organic buffers can include, for example, organic acids, carbonates, fatty acids, or a combination thereof. Examples of organic acid include citric, malic, lactic, acetic, ascorbic, tartaric, oxalic, benzoic, sorbic, or a combination thereof. Examples or carbonates can include sodium carbonate, sodium bicarbonate, zinc carbonate, potassium carbonate, potassium bicarbonate, or a combination thereof. Examples of fatty acid buffers can include lauric, myristic, stearic, oleic, palmitic, or a combination thereof. These buffers generally work in different pH ranges. For example, the organic acids work around pH 5 to 8; fatty acids around 6 to 10; carbonates around 8 to 10.5. These buffers can be used to allow targeting the right pH using a suitable base (e.g. NaOH) in a practical manner.

As another part of the investigation into potential solutions to meet the desired dioxane levels to maintain the ability to use fatty alcohol ethoxylates and ethoxylated sulfate surfactants in cleansing products, scrubbing of raw materials is investigated to see if it is sufficient to meet the level of dioxane desired. Samples of raw materials like, sodium laureth-1 sulfate (SLE1S), sodium laureth-3 sulfate (SLE3S), and sodium trideceth-2 sulfate (ST2S) at varying active levels are scrubbed using industry standard methods. The samples are at a pH of >12 (measured using a 10% aqueous solution of the raw material). An experimental body wash is formulated with a dioxane scrubbed sodium trideceth-2 sulfate surfactant. Those samples are then placed in an aging study to determine whether dioxane levels remain stable over time after scrubbing.

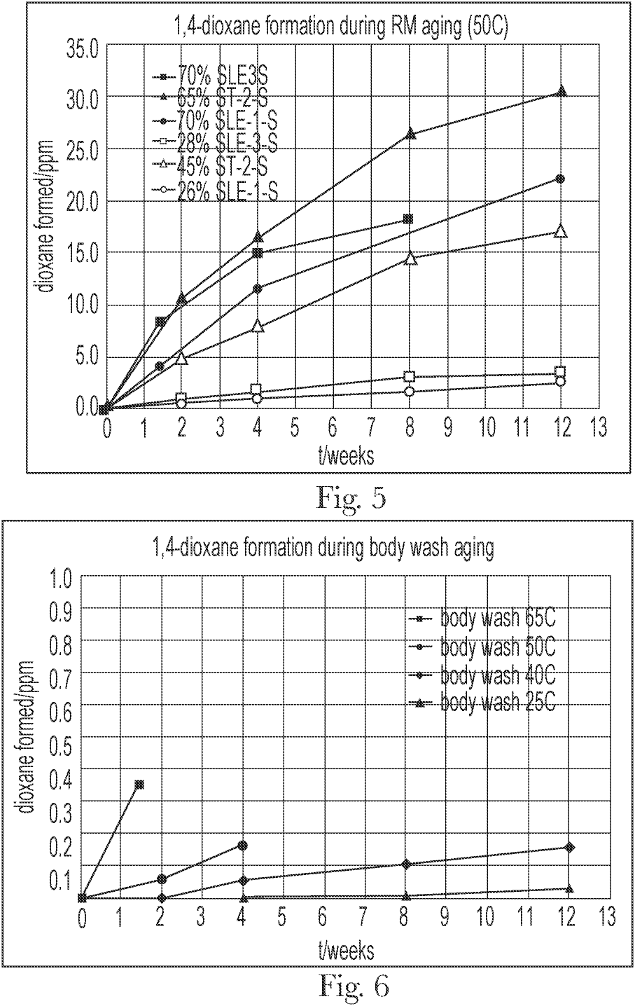

As can be seen from FIGS. 1, 2 and 5, three different raw materials (SLE1S, SLE3S, and ST2S) at various activities all showed an increase in dioxane over time. Thus, dioxane is produced in significant amounts in the dioxane scrubbed raw materials. The same is true for the body wash composition formulated with the scrubbed ST2S (9.6% active) at a pH of about 6. This is shown visually in FIG. 6.

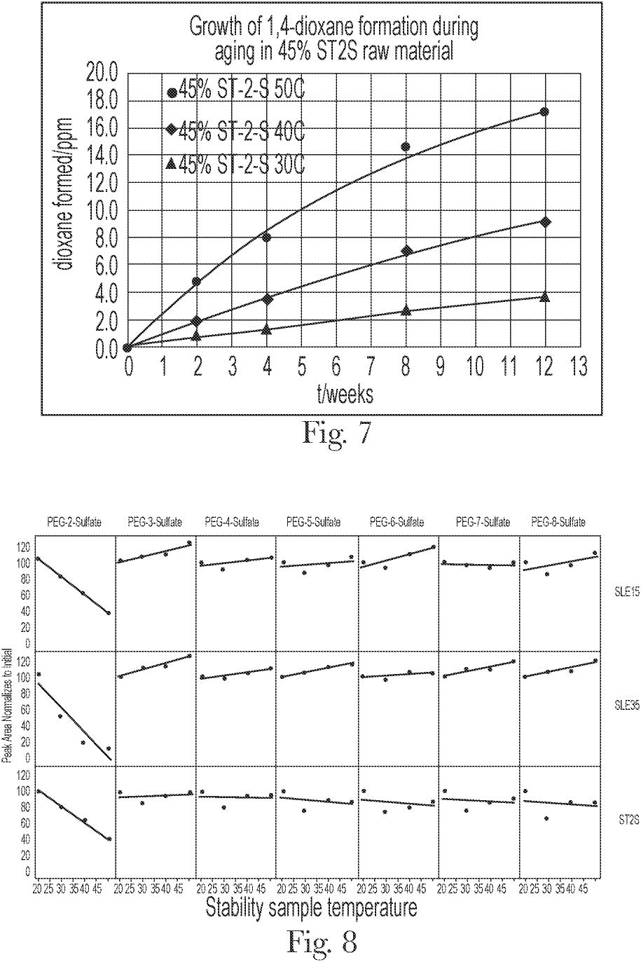

At longer storage times and higher temperatures, the dioxane generation rate is attenuated which suggests the dioxane could be formed from a component in the raw material which is then being exhausted. The characteristic pattern of regrowth is a plateau, as if exhausting an unknown reactant (see FIG. 7). Since the growth is likely caused by a component in the raw materials, the reaction byproducts of both the ethoxylation and sulfation reactions are reviewed to determine potential unknown reactants which could be allowing for the regrowth of dioxane. Primary byproducts, those from the ethoxylation reaction are predominantly free glycols, like ethylene glycol oligomer. The structure of an ethylene glycol oligomer is commonly expressed as H—(O—CH₂—CH₂)ₙ—OH, where n represents the average number of ethylene oxide units in the oligomer chain. n values of 2-30, for example, can be primary reaction byproducts. The n value of the oligomer chain may be, for example, 2 to about 25, 2 to about 20, 2 to about15, 2 to about 10, or 2 to about 5.

As explained above, these can undergo reaction during the sulfation process to produce secondary byproducts. Secondary byproducts are believed to include, for example, 1,4-dioxane; diethylene glycol monosulfate; diethylene glycol disulfate, and those byproducts formed as a result of the lysis of FAE and AES. The secondary byproducts are studied to determine which is the most likely to contribute to the increase in dioxane over time in an ethoxylated sulfate surfactant raw material.

Free glycols remain in fatty alcohol ethoxylates and the terminal hydroxyls are mostly sulfated in the falling film reactor at the same time as the fatty alcohol ethoxylate sulfation, so the transition states of the potential reactants are compared. As can be seen below in Table 2, diethylene glycol monosulfate is the only species which demonstrates a negative ΔG for the transition from diethylene glycol monosulfate to dioxane. Thus, it is the only reviewed species for which this reaction could be spontaneous at high pH and the most likely and most troubling byproduct for dioxane growth in an ethoxylated sulfate surfactant.

TABLE 2

| | Ea (kcal/mol) | ΔG (kcal/mol) | Relative reaction rate |
|---|---|---|---|
| diethylene glycol monosulfate | 18.9 | -25.2 | 1 * 10¹¹ |
| Free glycol monosulfates > 2 ethylene oxide units | 31.8 | +5.5 | 1.0 |

This is bolstered by the study of the concentration of free glycol(n) with n values of 2-8 after two months of aging at 25-50° C. Observations are plotted for 7 glycol sulfates, for three raw materials (SLE1S, ST2S, and SLE3S), and at four temperatures in FIG. 8. As can be seen from FIG. 8, the concentration of the tested glycol(n) monosulfates are relatively stable suggesting they are not being used up as a reaction product for the formation of dioxane. The one exception is diethylene glycol monosulfate which shows a significant decrease in concentration over the same time period at elevated temperature, compared to an ambient control.

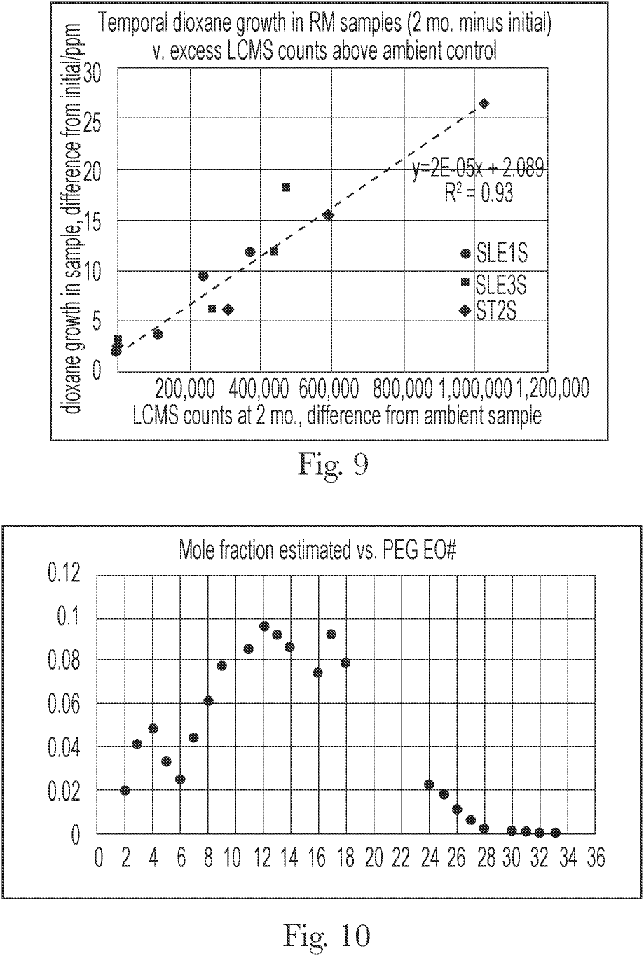

To further support the mechanism, we investigate the relationship between loss of diethylene glycol monosulfate and increase of dioxane in aging raw materials. To demonstrate this, the diethylene glycol monosulfate losses at each temperature compared to its ambient control is compared to the temporal increase in dioxane level in the same samples. This allows for the direct comparison of losses of diethylene glycol monosulfate with increases in dioxane over two months of aging. FIG. 9 compares dioxane growth over time for 3 surfactant raw materials at different temperatures (temperature not shown), to relative loss of diethylene glycol monosulfate at elevated temperatures compared to the ambient aged (control) sample for each surfactant. The relationship in FIG. 9 experimentally validates diethylene glycol monosulfate spontaneously converting to 1,4-dioxane.

Now that the likely culprit is identified, what is the solution for preventing it from forming unwanted dioxane? First, we look to how the diethylene glycol monosulfate is formed. It is believed some diethylene glycol monosulfate forms when primary byproducts of glycol from the ethoxylation reaction are sulfated during the sulfation process. We demonstrate this by sulfating a primary linear fatty alcohol mixed with 1 wt% polyethylene glycol 400 (PEG 400) mixed with the fatty alcohol, the PEG 400 representing free glycol, in an FFR. Results are compared to a similar fatty alcohol ethoxylate, laureth-1, sulfated under the same conditions, shown in Table 3. The added glycol contributes about 2% of the formation of diethylene glycol monosulfate, and about 30% of the dioxane formed during sulfation.

TABLE 3

| Feedstock | Free glycol | dioxane /ppm | diethylene glycol monosulfate/ppm |
|---|---|---|---|
| Linear fatty alcohol | none | 0 | 0 |
| Linear fatty alcohol | 1 wt% PEG 400 mixed in | 1.8 | 0.5 |
| Linear fatty alcohol ethoxylate (laureth-1) | about 0.5% as occurred | 6.8 | 25.7 |

So, if at least a portion of the glycols are removed from the fatty alcohol ethoxylate before it is used for the formation of an ethoxylated sulfate surfactant, then there should be less formation of dioxane itself, and dioxane precursors like diethylene glycol monosulfate and/or diethylene glycol disulfate and, correspondingly, less formation of dioxane during aging. The removal of at least some of the glycol byproduct would form a low glycol fatty alcohol ethoxylate, i.e. a fatty alcohol ethoxylate having less than about 0.25%, 0.2%, 0.15%, and/or 0.1% free glycol.

One way to remove at least a portion of the free glycol from a fatty alcohol ethoxylate is through the process of extraction. The extraction could be done, for example, with a solvent. The solvent could include, for example, water, ethanol and other alcohols, ethyl acetate and other esters, and other polar solvents. A solvent can be added at a level sufficient to allow the glycol to solubilize in the solvent. This could be, for example, from about 10% to about 100%, from about 10% to about 90%, 15% to about 80%, from about 15% to about 70%, from about 15% to about 60%, from about 25% to about 55%, and/or from about 30% to about 50%, by weight of the fatty alcohol ethoxylate material. The addition of the solvent is a balancing act between the desire to solubilize more of the glycol and the amount of alcohol ethoxylate that will be lost during the extraction process.

The extraction could further include the use of electrolytes such as salt. Examples of salt suitable for use during extraction are sodium chloride, potassium chloride, sodium citrate, potassium citrate, and combinations thereof. Salt can be added to help the separation of the solvent containing layer from the surfactant containing layer.

The extraction may be done at an elevated temperature. That temperature could be, for example, above the gelling point of the fatty alcohol ethoxylate / solvent mixture. When extracting the temperature may be, for example, from about 65° C. to about 90° C., from about 67° C. to about 88° C., from about 70° C. to about 90° C. 65° C. to about 75° C., from about 67° C. to about 70° C., about 70° C., and/or about 80° C.

When extraction is done with a solvent, the solvent may need to be removed from the fatty alcohol ethoxylate raw material. To remove solvent from the fatty alcohol ethoxylate raw material, the mixture can be dried, for example. The mixture can be dried utilizing an oven, air, heated air, vacuum dried, etc. An exemplary extraction and drying process can be found below.

A sample of a commercial ethoxylated alcohol surfactant which is 2-tridecyl alcohol ethoxylate is measured for one of its primary byproducts, glycol. As can be seen in FIG. 10, the sample contains many glycols. The largest concentration of which are PEG-12 and above, but PEG-10 through PEG-2 are also present. PEG-2 is present in the sample at above the level of detection but below the limit of quantitation and the value for PEG-2 in FIG. 10 is therefore an estimate. This sample is then put through an extraction process. First, the sample is dissolved in ethyl acetate. The glycols are then extracted with an aqueous solution of sodium chloride. The extraction is repeated three times. As can be seen in Table 4, the level of glycols was significantly reduced after just one wash and was undetectable after the second wash. The numbers are corrected for the differences in concentration. Thus, it is possible to remove at least a portion of the free glycol primary byproducts.

TABLE 4

| # of Washes | CAD peak area for glycols |
|---|---|
| 0 (Pre-wash) | 769 |
| 1 | 11 |
| 2 | Not detected |
| 3 | Not detected |

Thus, the amount of glycol in a reduced glycol fatty alcohol material can be, for example, from about 0 ppm to about 100 ppm, from about 0 ppm to about 90 ppm, from about 0 ppm to about 80 ppm, from about 0 ppm to about 70 ppm, from about 0 ppm to about 60 ppm, from about 0 ppm to about 50 ppm, from about 0 ppm to about 40 ppm, from about 0 ppm to about 30 ppm, from about 0 ppm to about 20 ppm, and/or from about 0 ppm to about 10 ppm. A reduced glycol fatty alcohol material is one where the amount of glycol byproduct has been reduced after formation of the glycol fatty alcohol material. Thus, a fatty alcohol material which falls within these ranges without having gone through a process to remove at least a portion of glycol byproduct would not be considered a reduced glycol sulfate fatty alcohol material. However, if that same material went through a process to remove at least a portion of glycol byproduct and that process resulted in a reduction of that byproduct, then the resulting material would be a reduced glycol fatty alcohol material. The amounts of glycol above are those where the surfactant activity has been adjusted to 100%.

The amount of glycol in a reduced glycol fatty alcohol material can be at least 10 % by weight less, from about 10% to about 100% less, from about 15% to about 95%, from about 15% to about 90%, from about 15% to about 85%, from about 20% to about 95%, from about 20% to about 90%, from about 20% to about 85%, from about 25% to about 95%, from about 25% to about 90%, from about 25% to about 85%, from about 25% to about 75%, from about 30% to about 95%, from about 30% to about 90%, from about 30% to about 85%, and/or from about 30% to about 75% by weight less than that of the fatty alcohol material prior to removal of at least a portion of the glycol.

In addition to the above, it is believed that removal of at least a portion of the glycols in an ethoxylated fatty alcohol prior to sulfation will result in an ethoxylated sulfate surfactant which has less glycol sulfate, aka. a reduced glycol sulfate ethoxylated sulfate surfactant. A laureth-1 surfactant can be sulfated to produce sodium laureth-1 sulfate (SLE1S). As can be seen from Table 5 below, an SLE1S surfactant made from laureth-1 in which at least a portion of ethylene glycol oligomer has not been removed prior to sulfation (control) contains more diethylene glycol monosulfate than the SLE1S sample made from the laureth-1 where at least a portion of ethylene glycol oligomer is removed prior to sulfation through extraction (washed).

TABLE 5

| sulfated control and washed laureth-1 | diethylene glycol monosulfate/ppm RM at 26% active (100% active) |
|---|---|
| SLE1S Control | 25.7 (99) |
| SLE1S from Washed Laureth-1 | 11 (42) |

Thus, a method of producing a reduced glycol sulfate ethoxylated sulfate surfactant can include, removing at least a portion of glycol in an ethoxylated fatty alcohol before it is sulfated to produce a reduced glycol sulfate ethoxylated sulfate surfactant. Another method of producing a reduced glycol sulfate ethoxylated surfactant can include sulfating a reduced glycol fatty alcohol.

A reduced glycol sulfate ethoxylated sulfate surfactant is one that has less glycol sulfate than if it had been produced with the ethoxylated fatty alcohol surfactant prior to removal of at least a portion of the glycol byproduct. The initial amount of glycol sulfate, before aging, can differ depending on the surfactant. For example, it is believed that an ethoxylated surfactant having only one ethoxylate group will have a lower initial amount of some diethylene glycol sulfates after the ethoxylation process than those ethoxylated surfactants having multiple ethoxylate units. In fact, it is believed the initial amount of diethylene glycol monosulfate increases with the addition of each ethoxylate group in the surfactant. Exemplary surfactants results are demonstrated in Table 6.

TABLE 6

| AES (activity) | diethylene glycol monosulfate RM/ppm (100% active) | diethylene glycol disulfate RM/ppm (100% active) |
|---|---|---|
| ST2S (65%) | 117 (180) | 329 (506) |
| SLE1S (26%) | 25.7 (99) | 223 (860) |

Since the initial level, before aging, of diethylene glycol monosulfate increases as the number of ethoxylate units increase, the ranges of amounts of glycol sulfate and diethylene glycol sulfate noted below can be adjusted to account for the differences in initial value to reflect an actual reduction from an ethoxylated surfactant without removal of at least a portion of the glycol byproduct. Thus, a surfactant which falls within these ranges without having gone through a process to remove at least a portion of glycol sulfate byproduct would not be considered a reduced glycol sulfate ethoxylated surfactant. However, if that same surfactant went through a process to remove at least a portion of glycol sulfate byproduct and that process resulted in a reduction of that byproduct, then the resulting surfactant would be a reduced glycol sulfate ethoxylated sulfate surfactant.

A reduced glycol sulfate ethoxylated sulfate surfactant can have less than about 100 ppm by weight of diethylene glycol monosulfate. The amount of diethylene glycol monosulfate in the reduced glycol ethoxylated sulfate surfactant is from about 0 ppm to about 100 ppm, from about 0 ppm to about 90 ppm, from about 0 ppm to about 80 ppm, from about 0 ppm to about 70 ppm, from about 0 ppm to about 60 ppm, from about 0 ppm to about 50 ppm, from about 0 ppm to about 40 ppm, from about 0 ppm to about 30 ppm, from about 0 ppm to about 20 ppm, or from about 0 ppm to about 10 ppm.

A reduced glycol sulfate ethoxylated sulfate surfactant can have less than about 300 ppm by weight of diethylene glycol disulfate. A reduced glycol sulfate ethoxylated sulfate surfactant can have less than about 250 ppm of diethylene glycol disulfate. A reduced glycol sulfate ethoxylated sulfate surfactant can have less than about 400 ppm of total diethylene glycol sulfates (the sum of the monosulfate and disulfate). The amount of diethylene glycol disulfate in the reduced glycol sulfate ethoxylated sulfate surfactant is from about 0 ppm to about 300 ppm, from about 0 ppm to about 275 ppm, from about 0 ppm to about 250 ppm, from about 0 ppm to about 200 ppm, from about 0 ppm to about 150 ppm, from about 0 ppm to about 100 ppm, from about 0 ppm to about 75 ppm, from about 0 ppm to about 50 ppm. The ppm amounts of glycol sulfate and diethylene glycol sulfate, above, are those where the surfactant activity has been adjusted to 100.

Another method of producing a reduced glycol sulfate ethoxylated surfactant can include sulfating a reduced glycol fatty alcohol with a less than 100% sulfation completion percentage. The completion percentage could be, for example, 99%, 98%, 97%, 96%, 95%, 94%, 93%, 92%, 91%, 90%, 89%, 88%, etc. Completion is reduced, for example, by utilizing lower mole ratios of SO₃ to Feedstock. The average molecular weight of the feedstock can be determined by measuring its hydroxyl value, prior to sulfation. Reducing SO₃ mole ratio can minimize excessive reactor (FFR) dioxane formation since SO₃ is catalytic for dioxane formation under reactor conditions (see Chemithon reference previously cited). The reduced glycol fatty alcohol can be produced in accordance with any of the methods previously described. For example, a reduced glycol fatty alcohol can be made by removing at least a portion of the glycol byproduct after the ethoxylation process. This can be done, for example, by extraction of at least a portion of the glycol byproduct. The glycol byproduct has been discussed above.

The reduced glycol sulfate surfactant can include, for example, sodium laureth(n) sulfate, and sodium trideceth(n) sulfate. n can have a value of 0.5 to about 4. For example, the n value can be from about 1 to about 3, about 1.8, and/or about 2.5.

In addition to providing solutions to help combat regrowth, the solutions can be used in combination with dioxane removal techniques to allow for both reduction of dioxane levels and the reduction of the potential for regrowth. For example, a method of producing a fatty alcohol ethoxylate material can include scrubbing a reduced glycol alcohol ethoxylate to remove at least a portion of 1,4-dioxane from the fatty alcohol ethoxylate material. This produces a scrubbed reduced glycol fatty alcohol ethoxylate. Another example includes a method of producing an ethoxylated sulfate surfactant can include scrubbing a reduced glycol sulfate ethoxylated surfactant to remove at least a portion of 1,4-dioxane from the surfactant. This results in a scrubbed reduced glycol sulfate ethoxylated surfactant. The scrubbing can be done by a process as discussed above or as is known in the art.

The reduced glycol alcohol ethoxylate and reduced glycol sulfate ethoxylated surfactant can be produced as discussed above. For example, after completion of the reaction to form a fatty alcohol ethoxylate material, at least a portion of the glycol byproduct is removed from the fatty alcohol ethoxylate material. The glycol may comprise, for example, an ethylene glycol oligomer. The ethylene glycol oligomer can comprise a chemical formula of HO(CH₂CH₂O)ₙH, wherein n is from 2 to about 30. At least a portion of the glycol may be removed, for example, by extraction.

The reduced glycol ethoxylated fatty alcohol surfactant, scrubbed glycol ethoxylated fatty alcohol surfactant, reduced glycol sulfate ethoxylated sulfate surfactant, and scrubbed reduced glycol sulfate ethoxylated sulfate surfactants can be used in a cleansing composition. More information on exemplary cleansing compositions is below.

Diethylene glycol monosulfate and diethylene glycol disulfate as used herein refer to the sodium salt and disodium salt, respectively. The molecular weight of the diethylene glycol monosulfate sodium salt is 208.2 g/mol and the diethylene glycol disulfate disodium salt is 310.2 g/mol.

### Cleansing Compositions

### 1. Personal Cleansing Compositions

Rinse-off personal cleansing compositions may come in many forms. For example, a personal cleansing composition may be in a liquid form and could be a body wash, shampoo, conditioning shampoo, shower gel, facial cleanser, cleansing milk, in shower body moisturizer, liquid hand soap, pet shampoo, shaving preparation, etc. Rinse-off personal cleansing compositions may also be in a solid form, like in a bar soap or a semi-solid form, like a paste or gel. Solid cleansing compositions can also be in many shapes and forms like a rectangle or in a powder or pellet form, for example. Additionally, solid and semi-solid forms may be combined with a substrate to form an article as described in more detail in U.S. Pat. Application Publication No. 2012/0246851; 2013/0043145; 2013/0043146; and 2013/0043147.

Rinse-off cleansing compositions generally include a cleansing ingredient, like a detersive surfactant and/or a soap. The amount of the cleansing ingredient will likely depend on form as better described below. Regardless of form, a cleansing composition may contain other ingredients like structurants, humectants, fatty acids, inorganic salts, antimicrobial agents, actives, preservatives, etc.

### A. Liquid Personal Cleansing Compositions

Exemplary liquid rinse-off personal cleansing compositions can include an aqueous carrier, which can be present at a level of from about 5% to about 95%, about 10% to about 90%; about 20% to about 80%, about 30% to about 75%; and/or about 40% to about 75%. The aqueous carrier may comprise water, or a miscible mixture of water and organic solvent. Non-aqueous carrier materials may also be employed.

Such rinse-off personal cleansing compositions may include one or more detersive surfactants. The detersive surfactant component can be included to provide cleaning performance to the product. The detersive surfactant component in turn comprises anionic detersive surfactant, zwitterionic detersive surfactant, amphoteric detersive surfactant, or a combination thereof. A representative, non-limiting, list of anionic surfactants includes anionic detersive surfactants for use in the compositions can include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In one example, the anionic surfactant can be sodium lauryl sulfate or sodium laureth sulfate. The concentration of the anionic surfactant component in the product can be sufficient to provide a desired cleaning and/or lather performance, and generally ranges from about 2% to about 50%.

Amphoteric detersive surfactants suitable for use in the rinse-off personal cleansing compositions are well known in the art, and include those surfactants broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoacetate, disodium lauroamphoacetate disodium cocodiamphoacetate, and mixtures thereof. Amphoacetates and diamphoacetates can also be used.

Zwitterionic detersive surfactants suitable for use in the rinse-off personal cleansing compositions are well known in the art, and include those surfactants broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine.

The personal cleansing composition can comprise one or more phases. Such personal cleansing compositions can include a cleansing phase and/or a benefit phase (i.e., a single- or multiphase composition). Each of a cleansing phase or a benefit phase can include various components. The cleansing phase and the benefit phase can be blended, separate, or a combination thereof. The cleansing phase and the benefit phase can also be patterned (e.g. striped).

The cleansing phase of a personal cleansing composition can include at least one surfactant. The cleansing phase may be an aqueous structured surfactant phase and be present at from about 5% to about 20%, by weight of the personal cleansing composition. Such a structured surfactant phase may include sodium trideceth(n) sulfate, hereinafter STnS, wherein n can define average moles of ethoxylation. n can range, for example, from about 0 to about 3; from about 0.5 to about 2.7, from about 1.1 to about 2.5, from about 1.8 to about 2.2, or n can be about 2. When n can be less than 3, STnS can provide improved stability, improved compatibility of benefit agents within the personal cleansing compositions, and increased mildness of the personal cleansing compositions, such described benefits of STnS are disclosed in U.S. Pat. No. 9,750,674.

The cleansing phase can also comprise at least one of an amphoteric surfactant and a zwitterionic surfactant. Suitable amphoteric or zwitterionic surfactants (in addition to those cited herein) can include, for example, those described in U.S. Pat. No. 5,104,646 and U.S. Pat. No. 5,106,609.

A cleansing phase can comprise a structuring system. A structuring system can comprise, optionally, a non-ionic emulsifier, optionally, from about 0.05% to about 5%, by weight of the personal cleansing composition, of an associative polymer; and an electrolyte.

The personal cleansing composition can be optionally free of sodium lauryl sulfate, hereinafter SLS, and can comprise at least a 70% lamellar structure. However, the cleansing phase could comprise at least one surfactant, wherein the at least one surfactant includes SLS. Suitable examples of SLS are described in U.S. Pat. Application Pub. No. 2010/0322878.

As noted herein, rinse-off personal cleansing compositions can also include a benefit phase. The benefit phase can be hydrophobic and/or anhydrous. The benefit phase can also be substantially free of surfactant. A benefit phase can also include a benefit agent. In particular, a benefit phase can comprise from about 0.1% to about 50%, by weight of the personal cleansing composition, of the benefit agent. The benefit phase may comprise less benefit agent, for example, from about 0.5% to about 20%, by weight of the personal cleansing composition, of the benefit agent. Examples of suitable benefit agents can include petrolatum, glyceryl monooleate, mineral oil, natural oils, and mixtures thereof. Additional examples of benefit agents can include water insoluble or hydrophobic benefit agents. Other suitable benefit agents are described in U.S. Pat. No. 9,750,674.

Non-limiting examples of glycerides suitable for use as hydrophobic skin benefit agents herein can include castor oil, safflower oil, corn oil, walnut oil, peanut oil, olive oil, cod liver oil, almond oil, avocado oil, palm oil, sesame oil, vegetable oils, sunflower seed oil, soybean oil, vegetable oil derivatives, coconut oil and derivatized coconut oil, cottonseed oil and derivatized cottonseed oil, jojoba oil, cocoa butter, and combinations thereof.

Non-limiting examples of alkyl esters suitable for use as hydrophobic skin benefit agents herein can include isopropyl esters of fatty acids and long chain esters of long chain (i.e. C10-C24) fatty acids, e.g., cetyl ricinoleate, non-limiting examples of which can include isopropyl palmitate, isopropyl myristate, cetyl riconoleate, and stearyl riconoleate. Other example can include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of polyglycerin fatty acid esters suitable for use as hydrophobic skin benefit agents herein can include decaglyceryl distearate, decaglyceryl diisostearate, decaglyceryl monomyriate, decaglyceryl monolaurate, hexaglyceryl monooleate, and combinations thereof.

The rinse-off personal cleansing composition may be applied by a variety of means, including by rubbing, wiping or dabbing with hands or fingers, or by means of an implement and/or delivery enhancement device. Non-limiting examples of implements include a sponge or sponge-tipped applicator, a mesh shower puff, a swab, a brush, a wipe (e.g., wash cloth), a loofah, and combinations thereof. Non-limiting examples of delivery enhancement devices include mechanical, electrical, ultrasonic and/or other energy devices. Employment of an implement or device may help delivery of the particulate antimicrobial agent to target regions, such as, for example, hair follicles and undulations that can exist in the underarm. The rinse-off cleansing product may be sold together with such an implement or device. Alternatively, an implement or device can be sold separately but contain indicium to indicate usage with a rinse-off cleansing product. Implements and delivery devices can employ replaceable portions (e.g., the skin interaction portions), which can be sold separately or sold together with the rinse-off cleansing product in a kit.

### B. Solid Personal Cleansing Compositions

As noted herein, personal cleansing compositions can take on numerous forms. One suitable form is that of a solid personal cleansing composition. Solid compositions can take many forms like powder, pellets, bars, etc. These forms will generally be described herein as bar soap, but it should be understood that the solid composition could be in another form or shape. One example of a bar soap personal cleansing composition can include from about 0.1% to about 35%, by weight of the personal cleansing composition, of water, from about 45% to about 99%, by weight of the personal cleansing composition, of soap, and from about 0.01% to about 5%, by weight of the personal cleansing composition, of a particulate antimicrobial agent. Another suitable antimicrobial bar soap can include, for example, from about 0.1% to about 30%, by weight of the personal cleansing composition, of water, from about 40% to about 99%, by weight of the personal cleansing composition, of soap, and from about 0.25% to about 3%, by weight of the personal cleansing composition, of a particulate antimicrobial agent.

Bar soap compositions can be referred to as conventional solid (i.e. non-flowing) bar soap compositions. Some bar soap composition comprise convention soap, while others contain synthetic surfactants, and still others contain a mix of soap and synthetic surfactant. Bar compositions may include, for example, from about 0% to about 45% of a synthetic anionic surfactant. An example of a suitable conventional soap can include milled toilet bars that are unbuilt (i.e. include about 5% or less of a water-soluble surfactancy builder).

A personal cleansing bar composition can include, for example from about 45% to about 99% or from about 50% to about 75%, by weight of the personal cleansing composition, of soap. Such soaps can include a typical soap, i.e., an alkali metal or alkanol ammonium salt of an alkane-or alkene monocarboxylic acid. Sodium, magnesium, potassium, calcium, mono-, di- and triethanol ammonium cations, or combinations thereof, can be suitable for a personal cleansing composition. The soap included in a personal cleansing composition can include sodium soaps or a combination of sodium soaps with from about 1% to about 25% ammonium, potassium, magnesium, calcium, or a mixture of these soaps. Additionally, the soap can be well-known alkali metal salts of alkanoic or alkenoic acids having from about 12 to about 22 carbon atoms or from about 12 to about 18 carbon atoms. Another suitable soap can be alkali metal carboxylates of alkyl or alkene hydrocarbons having from about 12 to about 22 carbon atoms. Additional suitable soap compositions are described in U.S. Pat. Application Pub. Nos. 2012/0219610 and 2008/0020959.

A personal cleansing composition can also include soaps having a fatty acid. For example, one bar soap composition could use from about 40% to about 95% of soluble alkali metal soap of C₈-C₂₄ or C₁₀-C₂₀ fatty acids. The fatty acid may, for example, have a distribution of coconut oil that can provide a lower end of a broad molecular weight range or a fatty acid distribution of peanut or rapeseed oil, or their hydrogenated derivatives, which can provide an upper end of the broad molecular weight range. Other such compositions can include a fatty acid distribution of tallow and/or vegetable oil. The tallow can include fatty acid mixtures that can typically have an approximate carbon chain length distribution of 2.5% C₁₄, 29% C₁₆, 23% C₁₈, 2% palmitoleic, 41.5% oleic, and 3% linoleic. The tallow can also include other mixtures with a similar distribution, such as fatty acids derived from various animal tallows and/or lard. In one example, the tallow can also be hardened (i.e., hydrogenated) such that some or all unsaturated fatty acid moieties can be converted to saturated fatty acid moieties.

Suitable examples of vegetable oil include palm oil, coconut oil, palm kernel oil, palm oil stearine, soybean oil, and hydrogenated rice bran oil, or mixtures thereof, since such oils can be among more readily available fats. One example of a suitable coconut oil can include a proportion of fatty acids having at least 12 carbon atoms of about 85%. Such a proportion can be greater when mixtures of coconut oil and fats such as tallow, palm oil, or non-tropical nut oils or fats can be used where principle chain lengths can be C₁₆ and higher. The soap included in a personal cleansing composition can be, for example, a sodium soap having a mixture of about 67-68% tallow, about 16-17% coconut oil, about 2% glycerin, and about 14% water.

Soap included in a personal cleansing composition can also be unsaturated in accordance with commercially acceptable standards. For example, a soap included in a personal cleansing composition could include unsaturation in a range of from about 37% to about 45% of saponified material.

Soaps included in a personal cleansing composition can be made, for example, by a classic kettle boiling process or modern continuous soap manufacturing processes wherein natural fats and oils such as tallow or coconut oil or their equivalents can be saponified with an alkali metal hydroxide using procedures well known to those skilled in the art. Soap can also be made by neutralizing fatty acids such as lauric (C₁₂), myristic (C₁₄), palmitic (C₁₆), or stearic (C₁₈) acids, with an alkali metal hydroxide or carbonate.

Soap included in a personal cleansing composition could also be made by a continuous soap manufacturing process. The soap could be processed into soap noodles via a vacuum flash drying process. One example of a suitable soap noodle comprises about 67.2% tallow soap, about 16.8% coconut soap, about 2% glycerin, and about 14% water, by weight of the soap noodle. The soap noodles can then be utilized in a milling process to finalize a personal cleansing composition.

### 2. Detergent Compositions for Fabric and Home Applications

The surfactant or surfactant intermediates described herein can be used for formulating detergent compositions for use fabric and home care applications, especially laundry and hard surface cleaning. Examples of laundry detergents include granular detergents as well as liquid laundry detergents, including low water liquid detergent compositions which are encapsulated in a water-soluble film to form a soluble unit dose article. Examples of hard surface cleaning detergent compositions include those suitable for cleaning floors, counter-tops, dishes and the like, with the surfactants and intermediates being particularly suited for hand-dishwashing detergent compositions.

Suitable laundry detergent compositions can comprise a non-soap surfactant, wherein the non-soap surfactant comprises an anionic non-soap surfactant and a non-ionic surfactant. The laundry detergent composition can comprise from 10% to 60%, or from 20% to 55% by weight of the laundry detergent composition of the non-soap surfactant. The non-soap anionic surfactant to non-ionic surfactant are from 1:1 to 20:1, from 1.5:1 to 17.5:1, from 2:1 to 15:1, or from 2.5:1 to 13: 1. Suitable non-soap anionic surfactants include linear alkylbenzene sulphonate, alkyl sulphate or a mixture thereof. The weight ratio of linear alkylbenzene sulphonate to alkyl sulphate can be from 1:2 to 9:1, from 1:1 to 7:1, from 1:1 to 5:1, or from 1:1 to 4:1. Suitable linear alkylbenzene sulphonates are C₁₀-C₁₆ alkyl benzene sulfonic acids, or C₁₁-C₁₄ alkyl benzene sulfonic acids. Suitable alkyl sulphate anionic surfactants include alkoxylated alkyl sulphates, non-alkoxylated alkyl sulphates, and mixture thereof. Preferably, the linear alkylbenzene sulphonates surfactant comprises greater than 50%, preferably greater than 60%, preferably greater than 70% C12, more preferably greater than 75% C₁₂ material. Suitable alkoxylated alkyl sulphate anionic surfactants include ethoxylated alkyl sulphate anionic surfactants which may be derived following the process of the invention or from a surfactant intermediate according to the invention. Suitable alkyl sulphate anionic surfactants include ethoxylated alkyl sulphate anionic surfactant with a mol average degree of ethoxylation of from 1 to 5, from 1 to 3, or from 2 to 3. The alkyl alkoxylated sulfate may have a broad alkoxy distribution or a peaked alkoxy distribution. The alkyl portion of the AES may include, on average, from 13.7 to about 16 or from 13.9 to 14.6 carbons atoms. At least about 50% or at least about 60% of the AES molecule may include an alkyl portion having 14 or more carbon atoms, preferably from 14 to 18, or from 14 to 17, or from 14 to 16, or from 14 to 15 carbon atoms. The alkyl sulphate anionic surfactant may comprise a non-ethoxylated alkyl sulphate and an ethoxylated alkyl sulphate wherein the mol average degree of ethoxylation of the alkyl sulphate anionic surfactant is from 1 to 5, from 1 to 3, or from 2 to 3. The alkyl fraction of the alkyl sulphate anionic surfactant can be derived from fatty alcohols, oxo-synthesized alcohols, Guerbet alcohols, or mixtures thereof. Preferred alkyl sulfates include optionally ethoxylated alcohol sulfates including 2-alkyl branched primary alcohol sulfates, especially 2-branched C₁₂₋₁₅ primary alcohol sulfates, linear primary alcohol sulfates especially linear C₁₂₋₁₄ primary alcohol sulfates, and mixtures thereof. The laundry detergent composition can comprise from 10% to 50%, or from 15% to 45%, or from 20% to 40%, or from 30% to 40% by weight of the laundry detergent composition of the non-soap anionic surfactant.

Suitable non-ionic surfactants can be selected from alcohol broad or narrow range alkoxylates, an oxo-synthesised alcohol alkoxylate, Guerbet alcohol alkoxylates, alkyl phenol alcohol alkoxylates, or a mixture thereof. The laundry detergent composition can comprise from 0.01% to 10%, from 0.01% to 8%, from 0.1% to 6%, or from 0.15% to 5% by weight of the liquid laundry detergent composition of a non-ionic surfactant.

The laundry detergent composition comprises from 1.5% to 20%, or from 2% to 15%, or from 3% to 10%, or from 4% to 8% by weight of the laundry detergent composition of soap, such as a fatty acid salt. Such soaps can be amine neutralized, for instance using an alkanolamine such as monoethanolamine.

The laundry detergent composition can comprises an adjunct ingredient selected from the group comprising builders including citrate, enzymes, bleach, bleach catalyst, dye, hueing dye, Leuco dyes, brightener, cleaning polymers including alkoxylated polyamines and polyethyleneimines, amphiphilic copolymers, soil release polymer, surfactant, solvent, dye transfer inhibitors, chelant, diamines, perfume, encapsulated perfume, polycarboxylates, structurant, pH trimming agents, antioxidants, antibacterial, antimicrobial agents, preservatives and mixtures thereof.

The laundry detergent composition can have a pH of from 2 to 10, or from 6.5 to 8.9, or from 7 to 8, wherein the pH of the laundry detergent composition is measured at a 10% product concentration in demineralized water at 20° C.

The liquid laundry detergent composition can be Newtonian or non-Newtonian, preferably non-Newtonian.

For liquid laundry detergent compositions, the composition can comprise from 5% to 99%, or from 15% to 90%, or from 25% to 80% by weight of the liquid detergent composition of water.

The liquid detergent composition can also be a low-water liquid detergent composition which comprises less than 15%, or less than 12% by weight of the liquid laundry detergent composition of water. Such laundry detergent compositions can comprise from 10% and 40%, or from 15% to 30% by weight of the liquid laundry detergent composition of a non-aqueous solvent selected from 1,2-propanediol, dipropylene glycol, tripropyleneglycol, glycerol, sorbitol, polyethylene glycol or a mixture thereof. Such liquid detergent compositions are particularly suitable for encapsulation in a water-soluble film in order to form a water-soluble unit dose laundry detergent article.

The at least one water-soluble film can be orientated to create at least one unit dose internal compartment, wherein the at least one unit dose internal compartment comprises the detergent composition. The water-soluble film can comprise polyvinyl alcohol polymer or copolymer, such as a blend of polyvinylalcohol polymers and/or polyvinylalcohol copolymers. The polyvinylalcohol polymers and/or polyvinylalcohol copolymers can be selected from sulphonated and carboxylated anionic polyvinylalcohol copolymers especially carboxylated anionic polyvinylalcohol copolymers, such as a blend of a polyvinylalcohol homopolymer and a carboxylated anionic polyvinylalcohol copolymer. The water soluble film can be supplied by Monosol under the trade references M8630, M8900, M8779, M8310.

Suitable liquid hand-dishwashing detergent compositions are typically aqueous, comprising from 50% to 90%, preferably from 60% to 75%, by weight of the total composition of water.

The hand-dishwashing composition can comprise from 5% to 50%, preferably from 8% to 45%, more preferably from 15% to 40%, by weight of the total composition of a surfactant system.

Such surfactant systems can comprise from 60% to 90%, more preferably from 70% to 80% by weight of the surfactant system of an anionic surfactant. Alkyl sulphated anionic surfactants are preferred, particularly those selected from the group consisting of: alkyl sulphate, alkyl alkoxy sulphate preferably alkyl ethoxy sulphate, and mixtures thereof. The alkyl sulphated anionic surfactant preferably has an average alkyl chain length of from 8 to 18, preferably from 10 to 14, more preferably from 12 to 14, most preferably from 12 to 13 carbon atoms. The alkyl sulphated anionic surfactant preferably has an average degree of alkoxylation preferably ethoxylation, of less than 5, preferably less than 3, more preferably from 0.5 to 2.0, most preferably from 0.5 to 0.9. Ethoxylated alkyl sulphate surfactants may be derived following the process of the invention / from the surfactant intermediate according to the invention. The alkyl sulphate anionic surfactant preferably has a weight average degree of branching of more than 10%, preferably more than 20%, more preferably more than 30%, even more preferably between 30% and 60%, most preferably between 30% and 50%. Suitable counterions include alkali metal cation earth alkali metal cation, alkanolammonium or ammonium or substituted ammonium, but preferably sodium. Suitable examples of commercially available alkyl sulphate anionic surfactants include, those derived from alcohols sold under the Neodol® brand-name by Shell, or the Lial®, Isalchem®, and Safol® brand-names by Sasol, or some of the natural alcohols produced by The Procter & Gamble Chemicals company.

The surfactant system can comprise from 0.1% to 20%, more preferably from 0.5% to 15% and especially from 2% to 10% by weight of the liquid hand dishwashing detergent composition of a co-surfactant. Preferred co-surfactants are selected from the group consisting of an amphoteric surfactant, a zwitterionic surfactant, and mixtures thereof. The anionic surfactant to the co-surfactant weight ratio can be from 1:1 to 8:1, preferably from 2:1 to 5:1, more preferably from 2.5:1 to 4:1. The co-surfactant is preferably an amphoteric surfactant, more preferably an amine oxide surfactant. Preferably, the amine oxide surfactant is selected from the group consisting of: alkyl dimethyl amine oxide, alkyl amido propyl dimethyl amine oxide, and mixtures thereof, most preferably C12-C14 alkyl dimethyl amine oxide. Suitable zwitterionic surfactants include betaine surfactants, preferably cocamidopropyl betaine.

Preferably, the surfactant system of the composition of the present invention further comprises from 1% to 25%, preferably from 1.25% to 20%, more preferably from 1.5% to 15%, most preferably from 1.5% to 5%, by weight of the surfactant system, of a non-ionic surfactant. Suitable nonionic surfactants can be selected from the group consisting of: alkoxylated non-ionic surfactant, alkyl polyglucoside (“APG”) surfactant, and mixtures thereof. Suitable alkoxylated non-ionic surfactants can be linear or branched, primary or secondary alkyl alkoxylated preferably alkyl ethoxylated non-ionic surfactants comprising on average from 9 to 15, preferably from 10 to 14 carbon atoms in its alkyl chain and on average from 5 to 12, preferably from 6 to 10, most preferably from 7 to 8, units of ethylene oxide per mole of alcohol. Most preferably, the alkyl polyglucoside surfactant has an average alkyl carbon chain length between 10 and 16, preferably between 10 and 14, most preferably between 12 and 14, with an average degree of polymerization of between 0.5 and 2.5 preferably between 1 and 2, most preferably between 1.2 and 1.6. C8-C16 alkyl polyglucosides are commercially available from several suppliers (e.g., Simusol® surfactants from Seppic Corporation; and Glucopon® 600 CSUP, Glucopon® 650 EC, Glucopon® 600 CSUP/MB, and Glucopon® 650 EC/MB, from BASF Corporation).

The liquid hand dishwashing detergent composition herein may optionally comprise a number of other adjunct ingredients such as builders (e.g., preferably citrate), chelants (e.g., preferably GLDA), conditioning polymers, cleaning polymers including polyalkoxylated polyalkylene imines, surface modifying polymers, soil flocculating polymers, sudsing polymers including EO-PO-EO triblock copolymers, grease cleaning amines including cyclic polyamines, structurants, emollients, humectants, skin rejuvenating actives, enzymes, carboxylic acids, scrubbing particles, bleach and bleach activators, perfumes, malodor control agents, pigments, dyes, opacifiers, beads, pearlescent particles, microcapsules, organic solvents, inorganic cations such as alkaline earth metals such as Ca/Mg-ions, antibacterial agents, preservatives, viscosity adjusters (e.g., salt such as NaCl, and other mono-, di- and trivalent salts) and pH adjusters and buffering means (e.g. carboxylic acids such as citric acid, HCl, NaOH, KOH, alkanolamines, phosphoric and sulfonic acids, carbonates such as sodium carbonates, bicarbonates, sesquicarbonates, borates, silicates, phosphates, imidazole and alike).

The pH of the hand-dishwashing detergent composition, measured as a 10% product concentration in demineralized water at 20° C., can be adjusted to between 3 and 14, more preferably between 4 and 13, more preferably between 6 and 12 and most preferably between 8 and 10. The hand-dishwashing composition can be Newtonian or non-Newtonian, preferably Newtonian. The composition can have a viscosity of from 10 mPa·s to 10,000 mPa·s, preferably from 100 mPa·s to 5,000 mPa·s, more preferably from 300 mPa·s to 2,000 mPa·s, or most preferably from 500 mPa·s to 1,500 mPa·s. The viscosity is measured at 20° C. with a Brookfield RT Viscometer using spindle 31 with the RPM of the viscometer adjusted to achieve a torque of between 40% and 60%.

### Examples

### 1. Extraction of Glycol Byproducts from Alcohol Ethoxylate

A drum of alcohol ethoxylate (~171 kg) is added to a vessel with 115 kg of water. The vessel is agitated at temperatures of 70-80° C. for 30 minutes prior to stopping agitation and allowing the aqueous and organic layers to gravity separate for between 1 and 4 hours before the bottom aqueous layer is drained off. The process can be repeated, for example 3 times, with 115 kg of water each time to thoroughly water wash the alcohol ethoxylate. At the end of the water washing, the alcohol ethoxylate is dried to help remove the added water maintaining the bulk temperature between 70-90° C. and pulling a vacuum down slowly to avoid foaming to ~15 mmHg. A nitrogen sparge through the alcohol ethoxylate can be added at the end of the drying with the vacuum to further pull out the moisture to <0.1 wt% with a target of 0.05 wt% or less. The final yield of the washed and dried alcohol ethoxylate is, for example, 97.5% of the starting alcohol ethoxylate weight.

### 2. Extraction of Polyethylene Glycol from Tridecyl Alcohol Ethoxylate- 2 (TDA-2)

600 g of TDA-2 are added to a 1 L open head glass reactor with a bottom drain valve. The contents are heated to 50° C. with mixing. 300 mL of water is added to the TDA-2. When the water is added at this temperature, the contents of the reactor can gel. If this happens, increase the temperature. Here, it is heated to 70° C. to keep everything fluid. The mixture is allowed to mix for 30 minutes. The mixing is then stopped and the contents are allowed to separate into layers. Once there are 2 layers, the bottom layer is drained. The bottom layer is the water layer and will contain the polyethylene glycol which is solubilized in the water. The top layer is washed again by adding in an additional 300 mL of water and repeating the process. This can be repeated as desired. Here, it is repeated for a total of 3 washes.

Once the washes are completed, the TDA-2 is dried to remove excess water. This is done by vacuum drying. The vacuum is slowly stepped down to avoid excess foaming until the desired vacuum level of about 17 mm Hg is reached. The vacuum is maintained until the TDA-2 appeared clear and there is no observable moisture being collected. If the final product has more moisture than desired, the time under vacuum drying can be increased. In addition, a nitrogen sparge line can be used in addition to the vacuum to drive out residual moisture.

3. Exemplary Personal Cleansing Compositions

| | Inventive Personal Cleansing Composition A | Inventive Personal Cleansing Composition B | Inventive Personal Cleansing Composition C | Inventive Personal Cleansing Composition D |
|---|---|---|---|---|
| Water | Q.S. | Q.S. | Q.S. | Q.S. |
| Reduced glycol sulfate Branched sodium trideceth-2-sulfate | 8.9% | 9.7% | - | - |
| Scrubbed Reduced glycol sulfate Branched sodium trideceth-2-sulfate | - | - | 8.9% | 9.7% |
| Cocamidopropyl betaine | 2.70% | 2.9% | 2.70% | 2.9% |
| Trideceth-3 Iconal TDA-3-Ethoxylated Tridecyl Alcohol | 1.66% | 1.66% | 1.66% | 1.66% |
| Sodium Chloride | 4.75% | 4.75% | 4.75% | 4.75% |
| Guar, Hydroxypropyl Trimonium Chloride, N-Hance CG-17 | 0.3% | 0.32% | 0.3% | 0.32% |
| Acrylates/C10-30 Alkyl Acrylates Cross Polymer | 0.03% | 0.03% | 0.03% | 0.03% |
| Xanthan gum | 0.19% | 0.2% | 0.19% | 0.2% |
| Petrolatum | - | 9.8% | - | 9.8% |
| Soybean Oil | 6.79% | - | 6.79% | - |
| Monoglyceryl Monooleate | 0.07% | 0.2% | 0.07% | 0.2% |
| Butylated hydroxyltoluene (BHT) | 0.14% | - | 0.14% | - |
| Disodium EDTA | 0.16% | 0.16% | 0.16% | 0.16% |
| Sodium Benzoate | 0.33% | 0.33% | 0.33% | 0.33% |
| Preservative | 0.03% | 0.03% | 0.03% | 0.03% |
| Perfume | 1.25% | 1.25% | 1.25% | 1.25% |
| pH | 5.70 | 5.70 | 5.70 | 5.70 |

Inventive Personal Cleansing Compositions A-D can be prepared by first premixing the Acrylates/C10-30 Alkyl Acrylates Cross Polymer, xanthan gum, and Trideceth-3 (Trideceth-3/polymer premix). Add water to a vessel, then add guar hydroxypropyltrimonium chloride and sodium chloride while mixing. Then, add reduced glycol sulfate sodium trideceth-2 sulfate, cocamidopropyl betaine, and the trideceth-3/polymer premix. Then add citric to adjust pH to 5.7. Then, add preservatives, and perfume. Keep mixing until homogeneous. Then, add the benefit agent(s) into the surfactant phase using a SpeedMixer and mix for 1 min at 2,000 rpm.

| Ingredient | Inventive Personal Cleansing Composition E | InventivePersonal Cleansing Composition F |
|---|---|---|
| Water | Q.S. | Q.S. |
| Reduced Glycol sulfate Sodium Laureth-3-Sulfate | 23.21 | - |
| Scrubbed reduced glycol sulfate Sodium Laureth-3-Sulfate | - | 23.21 |
| Sodium Lauryl Sulfate | 8.62 | 8.62 |
| Cocamidopropyl betaine | 3.33 | 3.33 |
| Sorbitol | 2.86 | 2.86 |
| Citric acid | 0.15 | 0.15 |
| Perfume | 0.60 | 0.60 |
| Sodium benzoate | 0.25 | 0.25 |
| Preservative | 0.05 | 0.05 |
| Sodium chloride | 2.00 | 2.00 |
| Antibacterial active | 0.005-5.0 | 0.005-5.0 |

Inventive Personal Cleansing Compositions E and F can be formed by the following process. DI water is added to a mixing vessel. Reduced glycol sulfate / scrubbed reduced glycol sulfate sodium laureth-3 sulfate, sodium lauryl sulfate, and cocamidopropyl betaine are added to the mixing vessel, followed by agitation of the vessel contents. Perfume is then added and mixed into the mixture for at least 10 minutes. Sodium benzoate is then added and allowed to dissolve into the mixture for at least 2 minutes. Citric acid is used to titrate the mixture until a pH of from about 6.5 to about 7.5 is reached, followed by the addition of preservative and then the antibacterial active. The mixture is mixed until homogeneous. Sodium chloride is then added and allowed to dissolve into the mixture for at least 2 minutes. DI water and/or sodium chloride are then added to adjust the viscosity of the mixture, which has a target range of 4,500-7,500 cP.

### 4. Inventive Laundry Formulations

The following are exemplary liquid laundry detergent formulations. The ethoxylated alkyl sulphate surfactant may be derived following the process of the invention / from the surfactant intermediate according to the invention.

| | | | |
|---|---|---|---|
| Raw Material | Laundry Composition A %wt | Laundry Composition B %wt | Laundry Composition C %wt |
| Branched Alkyl Sulfate | 0.0 | 5.3 | 0.0 |
| Sodium Lauryl Sulfate | 0.0 | 3.0 | 0.0 |
| linear alkylbenzene sulfonate | 18.0 | 5.0 | 6.0 |
| AE3S Ethoxylated alkyl sulphate with an average degree of ethoxylation of 3 | 5.0 | 0.0 | 1.3 |
| C25AES Ethoxylated alkyl sulphate with an average degree of ethoxylation of 2.5¹ | 0.0 | 3.0 | 1.4 |
| amine oxide | 0.7 | 1.0 | 0.4 |
| C24 alkyl ethoxylate (EO7) | 8.4 | 0.0 | 12.9 |
| C24 alkyl ethoxylate (EO9) | 0.0 | 8.7 | 0.0 |
| C45 alkyl ethoxylate (EO7) | 0.0 | 2.7 | 0.0 |
| citric acid | 2.9 | 2.3 | 0.7 |
| palm kernel fatty acid | 0.0 | 1.0 | 0.0 |
| topped kernel fatty acid | 2.9 | 0.0 | 2.3 |
| Mannanase | 0.0017 | 0.0017 | 0.0017 |
| Pectawash | 0.00342 | 0.00342 | 0.00342 |
| Amylase | 0.00766 | 0.00766 | 0.00766 |
| Protease | 0.07706 | 0.07706 | 0.07706 |
| Nuclease³ | 0.010 | 0.01 | 0.01 |
| Sodium tetraborate | 0.0 | 1.7 | 0.0 |
| MEA-Boric Acid Salt | 0.0 | 0.0 | 0.8 |
| calcium/sodium formate | 0.0 | 0.04 | 0.01 |
| Sodium/Calcium Chloride | 0.04 | 0.02 | 0.03 |
| Ethoxylated polyethyleneimine² | 0.0 | 2.0 | 1.1 |
| Amphiphilic graft copolymer | 1.5 | 0.0 | 0.0 |
| Ethoxylated-Propoxylated polyethyleneimine | 0.0 | 2.0 | 0.8 |
| Zwitterionic polyamine | 0.5 | 0.0 | 0.0 |
| Nonionic polyester terephthalate | 1.0 | 1.0 | 1.0 |
| DTPA | 0.0 | 0.1 | 0.2 |
| EDDS | 0.1 | 0.0 | 0.0 |
| GLDA | 0.4 | 0.3 | 0.1 |
| MGDA | 0.2 | 0.0 | 0.0 |
| diethylene triamine penta(methyl phosphonic) acid (DTPMP) | 1.1 | 0.0 | 0.0 |
| Fluorescent Brightener⁸ | 0.06 | 0.22 | 0.03 |
| Ethanol | 0.7 | 1.9 | 0.0 |
| propylene glycol | 5.5 | 5.5 | 0.33 |
| Sorbitol | 0.01 | 0.01 | 0.0 |
| monoethanolamine | 0.2 | 0.2 | 0.6 |
| DETA | 0.1 | 0.08 | 0.0 |
| Antioxidant 1 | 0.0 | 0.1 | 0.1 |
| Antioxidant 2 | 0.1 | 0.0 | 0.0 |
| Hygiene Agent | 0.0 | 0.0 | 0.05 |
| NaOH | 4.7 | 4.7 | 1.1 |
| NaCS | 3.2 | 1.7 | 3.2 |
| Hydrogenated Castor Oil | 0.2 | 0.1 | 0.12 |
| aesthetic dye | 0.10 | 0.01 | 0.006 |
| Leuco dye | 0.05 | 0.01 | 0.0 |
| Perfume | 2.0 | 1.3 | 0.5 |
| Perfume microcapsules | 0.5 | 0.05 | 0.1 |
| silicone antifoam⁷ | 0.02 | 0.01 | 0.0 |
| phenyloxyethanol | 0.002 | 0.01 | 0.0 |
| Hueing dye | 0.01 | 0.1 | 0.05 |
| water & miscellaneous | balance | balance | balance |

1 C12-15EO2.5S AlkylethoxySulfate where the alkyl portion of AES includes from about 13.9 to 14.6 carbon atoms
2 PE-20 commercially available from BASF
3 Nuclease enzyme is as claimed in co-pending European application 19219568.3
4 Antioxidant 1 is 3,5-bis(1,1-dimethylethyl)-4-hydroxybenzenepropanoic acid, methyl ester [6386-38-5]
5 Antioxidant 2 is Tinogard TS commercially available from BASF
6 Hygiene Agent is agent is Tinosan HP 100 commercially available from BASF
7 Dow Corning supplied antifoam blend 80-92% ethylmethyl, methyl(2-phenyl propyl)siloxane; 5-14% MQ Resin in octyl stearate a 3-7% modified silica.
8 Fluorescent Brightener is disodium 4,4'-bis{ [4-anilino-6-morpholino-s-triazin-2-yl]-amino}-2,2'-stilbenedisulfonate or 2,2'-([1,1'-Biphenyl]-4,4'-diyldi-2,1-ethenediyl)bis-benzenesulfonic acid disodium salt.

### 5. Inventive Water Soluble Unit Dose Formulation

The following is an exemplary water soluble unit dose formulations. The ethoxylated alkyl sulphate surfactant may be derived following the process of the invention / from the surfactant intermediate according to the invention. The composition can be part of a single chamber water soluble unit dose article or can be split over multiple compartments resulting in below “averaged across compartments” full article composition.

| Ingredients | Unit Dose Composition 1 (wt%) |
|---|---|
| Fatty alcohol ethoxylate non-ionic surfactant, C₁₂₋₁₄ average degree of ethoxylation of 7 | 3.8 |
| Lutensol XL100 | 0.5 |
| Linear C₁₁₋₁₄ alkylbenzene sulphonate | 24.6 |
| AE3S Ethoxylated alkyl sulphate with an average degree of ethoxylation of 3 | 12.5 |
| Citric acid | 0.7 |
| Palm Kernel Fatty acid | 5.3 |
| Nuclease enzyme (wt% active protein) | 0.01 |
| Protease enzyme (wt% active protein) | 0.07 |
| Amylase enzyme (wt% active protein) | 0.005 |
| Xyloglucanese enzyme (wt% active protein) | 0.005 |
| Mannanase enzyme (wt% active protein) | 0.003 |
| Ethoxylated polyethyleneimine | 1.6 |
| Amphiphilic graft copolymer | 2.6 |
| Zwitterionic polyamine | 1.8 |
| Anionic polyester terephthalate | 0.6 |
| HEDP | 2.2 |
| Brightener 49 | 0.4 |
| Silicone anti-foam | 0.3 |
| Hueing dye | 0.05 |
| 1,2 PropaneDiol | 12.3 |
| Glycerine | 4.7 |
| DPG (DiPropyleneGlycol) | 1.7 |
| TPG (TriPropyleneGlycol) | 0.1 |
| Sorbitol | 0.1 |
| Monoethanolamine | 10.2 |
| K2SO3 | 0.4 |
| MgC12 | 0.3 |
| water | 10.8 |
| Hydrogenated castor oil | 0.1 |
| Perfume | 2.1 |
| Aesthetic dye & Minors | Balance to 100 |
| pH (10% product concentration in demineralized water at 20° C.) | 7.4 |

### 6. Inventive Liquid Hand Dishwashing Detergent

The following is an exemplary liquid hand dishwashing detergent formulation. The formulation can be made through standard mixing of the individual components. The ethoxylated alkyl sulphate surfactant may be derived following the process of the invention / from the surfactant intermediate according to the invention

| As 100% active | Inventive Hand Dishwashing Composition 1 wt% |
|---|---|
| C1213AE0.6S anionic surfactant (Avg. branching: 37,84%) | 19.6 |
| C1214 dimethyl amine oxide | 6.5 |
| Alcohol ethoxylate nonionic surfactant (Neodol 91/8) | 1.0 |
| Alkoxylated polyethyleneimine (PEI600EO24PO16) | 0.2 |
| ethanol | 2.4 |
| NaCl | 0.7 |
| Polypropyleneglycol (MW2000) | 0.9 |
| Water + Minor ingredients (perfume, dye, preservatives) | Balance to 100 |
| pH (at 10% product concentration in demineralized water - with NaOH trimming) | 9.0 |

### Combinations

A. A method of producing a reduced glycol fatty alcohol ethoxylate material comprising, removing at least a portion of glycol in the fatty alcohol ethoxylate material after formation of the fatty alcohol ethoxylate material.

B. The method of paragraph A, wherein the glycol comprises an ethylene glycol oligomer.

C. The method of paragraph A or B, wherein the ethylene glycol oligomer comprises a chemical formula of HO(CH₂CH₂O)ₙH, wherein n is from 2 to about 30, preferably from 2 to about 25, more preferably from 2 to about 15, even more preferably from 2 to about 10, and most preferably from 2 to about 5.

D. The method of any of paragraphs A-C, wherein at least a portion of the glycol is removed by extracting the glycol from the fatty alcohol ethoxylate material.

E. The method of paragraph D, wherein at least a portion of the glycol is extracted with water.

F. The method of paragraphs D or E, wherein the glycol is extracted at a temperature above the gelling point of the fatty alcohol ethoxylate water mixture.

G. The method of paragraph F, wherein the extraction temperature is from about 65° C. to about 90° C., preferably from about 67° C. to about 88° C., or more preferably from about 70° C. to about 85° C.

H. The method of paragraph F, wherein the glycol is extracted at a temperature of about 70° C.

I. The method of any of paragraphs D-H, wherein at least a portion of the glycol is extracted with a combination of water and sodium chloride.

J. The method of any of paragraphs D-I, wherein the reduced glycol fatty alcohol material is concentrating by drying off at least a portion of the water.

K. The method of any of paragraphs A-J, wherein the amount of glycol in the reduced glycol fatty alcohol material is from about 0 ppm to about 100 ppm, preferably from about 0 ppm to about 90 ppm, more preferably from about 0 ppm to about 80 ppm, even more preferably from about 0 ppm to about 70 ppm, even more preferably from about 0 ppm to about 60 ppm, even more preferably from about 0 ppm to about 50 ppm, even more preferably from about 0 ppm to about 40 ppm, even more preferably from about 0 ppm to about 30 ppm, even more preferably from about 0 ppm to about 20 ppm, and most preferably from about 0 ppm to about 10 ppm.

L. The method of any of paragraphs A-K, wherein the amount of glycol in the reduced glycol fatty alcohol material is at least 10 % by weight less, preferably from about 10% to about 100% less, from about 15% to about 95% less, from about 15% to about 90% less, from about 15% to about 85% less, from about 20% to about 95% less, from about 20% to about 90% less, from about 20% to about 85 less, from about 25% to about 95% less, from about 25% to about 90%, from about 25% to about 85%, from about 25% to about 75%, from about 30% to about 95%, from about 30% to about 90%, from about 30% to about 85%, and/or from about 30% to about 75% by weight less than that of the fatty alcohol material prior to removal of at least a portion of the glycol.

M. The method of any of paragraphs A-L, wherein the fatty alcohol ethoxylate material comprises laureth-1, laureth-2, laureth-3, trideceth-1, trideceth-2, trideceth-2.5, trideceth-3, or a combination thereof.

N. A method of producing a fatty alcohol ethoxylate material comprising, scrubbing a reduced glycol fatty alcohol material as described in any of paragraphs A-M to remove at least a portion of 1,4-dioxane.

O. A method of reducing the amount of ethylene glycol oligomer in an alcohol ethoxylate material, comprising, extracting ethylene glycol oligomer from the alcohol ethoxylate material.

P. The method of paragraph O, wherein the glycol comprises an ethylene glycol oligomer.

Q. The method of paragraph O or P, wherein the ethylene glycol oligomer comprises a chemical formula of HO(CH₂CH₂O)ₙH, wherein n is from 2 to about 30, preferably 2 to about 25, more preferably 2 to about 15, even more preferably 2 to about 10, and most preferably 2 to about 5.

R. The method of any of paragraphs O-Q, wherein at least a portion of the glycol is removed by extracting the glycol from the fatty alcohol ethoxylate material.

S. The method of paragraph R, wherein at least a portion of the glycol is extracted with water.

T. The method of paragraphs R or S, wherein the glycol is extracted at a temperature above the gelling point of the fatty alcohol ethoxylate water mixture.

U. The method of paragraph S, wherein the extraction temperature is from about 65° C. to about 90° C., preferably from about 67° C. to about 88° C., or more preferably from about 70° C. to about 85° C.

V. The method of paragraph S, wherein the glycol is extracted at a temperature of about 70° C.

W. The method of any of paragraphs R-V, wherein at least a portion of the glycol is extracted with a combination of water and sodium chloride.

X. The method of any of paragraphs R-W, wherein the reduced glycol fatty alcohol material is concentrating by drying off at least a portion of the water.

Y. The method of any of paragraphs O-X, wherein the amount of glycol in the reduced glycol fatty alcohol material is from about 0 ppm to about 100 ppm, preferably from about 0 ppm to about 90 ppm, more preferably from about 0 ppm to about 80 ppm, even more preferably from about 0 ppm to about 70 ppm, even more preferably from about 0 ppm to about 60 ppm, even more preferably from about 0 ppm to about 50 ppm, even more preferably from about 0 ppm to about 40 ppm, even more preferably from about 0 ppm to about 30 ppm, even more preferably from about 0 ppm to about 20 ppm, and most preferably from about 0 ppm to about 10 ppm.

Z. The method of any of paragraphs O-Y, wherein the amount of glycol in the reduced glycol fatty alcohol material is at least 10 % by weight less, preferably from about 10% to about 100% less, from about 15% to about 95%, from about 15% to about 90%, from about 15% to about 85%, from about 20% to about 95%, from about 20% to about 90%, from about 20% to about 85%, from about 25% to about 95%, from about 25% to about 90%, from about 25% to about 85%, from about 25% to about 75%, from about 30% to about 95%, from about 30% to about 90%, from about 30% to about 85%, and/or from about 30% to about 75% by weight less than that of the fatty alcohol material prior to removal of at least a portion of the glycol.

AA. The method of any of paragraphs O-Z, wherein the fatty alcohol ethoxylate material comprises laureth-1, laureth-2, laureth-3, trideceth-1, trideceth-2, trideceth-2.5, trideceth-3, or a combination thereof.

A2. A method of producing a reduced glycol sulfate ethoxylated sulfate surfactant comprising, removing at least a portion of glycol in an ethoxylated fatty alcohol raw material before the ethoxylated fatty alcohol raw material is sulfated to produce the reduced glycol sulfate ethoxylated sulfate surfactant.

B2. The method of paragraph A2, wherein the glycol comprises an ethylene glycol oligomer.

C2. The method of paragraph A2 or B2, wherein the ethylene glycol oligomer comprises a chemical formula of HO(CH₂CH₂O)ₙH, wherein n is from 2 to about 30, preferably from 2 to about 25, more preferably from 2 to about 15, even more preferably from 2 to about 10, and most preferably from 2 to about 5.

D2. The method of any of paragraphs A2-C2, wherein at least a portion of the glycol is removed by extracting the glycol from the ethoxylated fatty alcohol raw material.

E2. The method of paragraph D2, wherein at least a portion of the glycol is extracted with water.

F2. The method of paragraphs D2 or E2, wherein the glycol is extracted at a temperature above the gelling point of the fatty alcohol ethoxylate water mixture.

G2. The method of paragraph F2, wherein the extraction temperature is from about 65° C. to about 90° C., preferably from about 67° C. to about 88° C., or more preferably from about 70° C. to about 85° C.

H2. The method of paragraph F2, wherein the glycol is extracted at a temperature of about 70° C.

I2. The method of any of paragraphs D2-H2, wherein at least a portion of the glycol is extracted with a combination of water and sodium chloride.

J2. The method of any of paragraphs D2-I2, wherein after extraction the ethoxylated fatty alcohol raw material is concentrated by drying off at least a portion of the water.

K2. The method of any of paragraphs A2-J2, wherein the amount of glycol in the ethoxylated fatty alcohol raw material after removal of at least a portion of the glycol is from about 0 ppm to about 100 ppm, preferably from about 0 ppm to about 90 ppm, more preferably from about 0 ppm to about 80 ppm, even more preferably from about 0 ppm to about 70 ppm, even more preferably from about 0 ppm to about 60 ppm, even more preferably from about 0 ppm to about 50 ppm, even more preferably from about 0 ppm to about 40 ppm, even more preferably from about 0 ppm to about 30 ppm, even more preferably from about 0 ppm to about 20 ppm, and most preferably from about 0 ppm to about 10 ppm.

L2. The method of any of paragraphs A2-K2, wherein the amount of glycol in the ethoxylated fatty alcohol raw material after removal of at least a portion of the glycol is at least 10 % by weight less, preferably from about 10% to about 100% less, from about 15% to about 95% less, from about 15% to about 90% less, from about 15% to about 85% less, from about 20% to about 95% less, from about 20% to about 90% less, from about 20% to about 85 less, from about 25% to about 95% less, from about 25% to about 90%, from about 25% to about 85%, from about 25% to about 75%, from about 30% to about 95%, from about 30% to about 90%, from about 30% to about 85%, and/or from about 30% to about 75% by weight less than that of the ethoxylated fatty alcohol raw material prior to removal of at least a portion of the glycol.

M2. The method of any of paragraphs A2-L2, wherein the reduced glycol sulfate ethoxylated sulfate surfactant comprises sodium laureth-1 sulfate, sodium laureth-2 sulfate, sodium laureth-3 sulfate, sodium trideceth-1 sulfate, sodium trideceth-2 sulfate, sodium trideceth-2.5 sulfate, sodium trideceth-3 sulfate, or a combination thereof.

N2. The method of any of paragraphs A2-M2, wherein the amount of glycol sulfate by weight in the reduced glycol sulfate ethoxylated surfactant is 100 ppm by weight or less.

O2. The method of any of paragraphs A2-N2, wherein the reduced glycol sulfate ethoxylated sulfate surfactant has 100 ppm or less by weight of the diethyleneglycol monosulfate.

P2. The method of any of paragraphs A2-O2, wherein the amount of glycol sulfate in the reduced glycol ethoxylated sulfate surfactant is from about 0 ppm to about 100 ppm, from about 0 ppm to about 90 ppm, from about 0 ppm to about 80 ppm, from about 0 ppm to about 70 ppm, from about 0 ppm to about 60 ppm, from about 0 ppm to about 50 ppm, from about 0 ppm to about 40 ppm, from about 0 ppm to about 30 ppm, from about 0 ppm to about 20 ppm, or from about 0 ppm to about 10 ppm.

Q2. The method of any of paragraphs A2-P2, wherein the sulfation reaction is taken to less than 100% completion.

R2. The method of paragraph Q2, wherein the sulfation reaction is taken to 97%, 94%, or 90% completion.

S2. Use of a reduced glycol ethoxylated material to produce a reduced glycol sulfate ethoxylated sulfate surfactant.

T2. A method of producing an ethoxylated sulfate surfactant comprising, scrubbing a reduced glycol sulfate ethoxylated sulfate surfactant as described in any of paragraphs A-R to remove at least a portion of 1,4-dioxane.

U2. A method of producing a reduced glycol sulfate ethoxylated surfactant comprising sulfating a reduced glycol fatty alcohol to produce the reduced glycol sulfate ethoxylated sulfate surfactant.

V2. The method of paragraph U2, wherein the glycol comprises an ethylene glycol oligomer.

W2. The method of paragraph U2 or V2, wherein the ethylene glycol oligomer comprises a chemical formula of HO(CH₂CH₂O)ₙH, wherein n is from 2 to about 30, preferably from 2 to about 25, more preferably from 2 to about 15, even more preferably from 2 to about 10, and most preferably from 2 to about 5.

X2. The method of any of paragraphs U2-W2, wherein at least a portion of the glycol is removed by extracting the glycol from an ethoxylated fatty alcohol raw material.

Y2. The method of paragraph X2, wherein at least a portion of the glycol is extracted with water.

Z2. The method of paragraphs X2 or Y2, wherein the glycol is extracted at a temperature above the gelling point of the fatty alcohol ethoxylate water mixture.

AA2. The method of paragraph Z2, wherein the extraction temperature is from about 65° C. to about 90° C., preferably from about 67° C. to about 88° C., or more preferably from about 70° C. to about 85° C.

BB2. The method of paragraph Z2, wherein the glycol is extracted at a temperature of about 70° C.

CC2. The method of any of paragraphs X2-BB2, wherein at least a portion of the glycol is extracted with a combination of water and sodium chloride.

DD2. The method of any of paragraphs X2-CC2, wherein after extraction the ethoxylated fatty alcohol raw material is concentrated by drying off at least a portion of the water.

EE2. The method of any of paragraphs U2-DD2, wherein the amount of glycol in the reduced glycol fatty alcohol is from about 0 ppm to about 100 ppm, preferably from about 0 ppm to about 90 ppm, more preferably from about 0 ppm to about 80 ppm, even more preferably from about 0 ppm to about 70 ppm, even more preferably from about 0 ppm to about 60 ppm, even more preferably from about 0 ppm to about 50 ppm, even more preferably from about 0 ppm to about 40 ppm, even more preferably from about 0 ppm to about 30 ppm, even more preferably from about 0 ppm to about 20 ppm, and most preferably from about 0 ppm to about 10 ppm.

FF2. The method of any of paragraphs U2-EE2, wherein the amount of glycol in the reduced glycol fatty alcohol is at least 10 % by weight less, preferably from about 10% to about 100% less, from about 15% to about 95% less, from about 15% to about 90% less, from about 15% to about 85% less, from about 20% to about 95% less, from about 20% to about 90% less, from about 20% to about 85 less, from about 25% to about 95% less, from about 25% to about 90%, from about 25% to about 85%, from about 25% to about 75%, from about 30% to about 95%, from about 30% to about 90%, from about 30% to about 85%, and/or from about 30% to about 75% by weight less than that of an ethoxylated fatty alcohol raw material prior to removal of at least a portion of the glycol.

GG2. The method of any of paragraphs U2-FF2, wherein the reduced glycol sulfate ethoxylated sulfate surfactant comprises sodium laureth-1 sulfate, sodium laureth-2 sulfate, sodium laureth-3 sulfate, sodium trideceth-1 sulfate, sodium trideceth-2 sulfate, sodium trideceth-2.5 sulfate, sodium trideceth-3 sulfate, or a combination thereof.

HH2. The method of any of paragraphs U-GG2, wherein the amount of glycol sulfate by weight in the reduced glycol sulfate ethoxylated surfactant is 100 ppm by weight or less.

II2. The method of any of paragraphs U2-HH2, wherein the reduced glycol sulfate ethoxylated sulfate surfactant has 100 ppm or less by weight of diethyleneglycol monosulfate.

JJ2. The method of any of paragraphs U2-II2, wherein the amount of glycol sulfate in the reduced glycol ethoxylated sulfate surfactant is from about 0 ppm to about 100 ppm, from about 0 ppm to about 90 ppm, from about 0 ppm to about 80 ppm, from about 0 ppm to about 70 ppm, from about 0 ppm to about 60 ppm, from about 0 ppm to about 50 ppm, from about 0 ppm to about 40 ppm, from about 0 ppm to about 30 ppm, from about 0 ppm to about 20 ppm, or from about 0 ppm to about 10 ppm.

KK2. The method of any of paragraphs U2-JJ2, wherein the sulfation reaction is taken to less than 100% completion.

LL2. The method of paragraph KK2, wherein the sulfation reaction is taken to 97%, 94%, or 90% completion.

MM2. A method of producing an ethoxylated sulfate surfactant comprising, scrubbing a reduced glycol sulfate ethoxylated sulfate surfactant as described in any of paragraphs U-LL to remove at least a portion of 1,4-dioxane.

A3. A surfactant comprises an ethoxylated fatty alcohol with 100 ppm or less by weight of free glycol.

B3. The surfactant of paragraph A3, wherein the free glycol comprises an ethylene glycol oligomer.

C3. The surfactant of paragraph A3 or B3, wherein the ethylene glycol oligomer comprises a chemical formula of HO(CH₂CH₂O)ₙH, wherein n is from 2 to about 30, preferably from 2 to about 25, more preferably from 2 to about 15, even more preferably from 2 to about 10, and most preferably from 2 to about 5.

D3. The surfactant of any of paragraphs A3-C3, wherein the amount of free glycol in the ethoxylated fatty alcohol is from about 0 ppm to about 100 ppm, preferably from about 0 ppm to about 90 ppm, more preferably from about 0 ppm to about 80 ppm, even more preferably from about 0 ppm to about 70 ppm, even more preferably from about 0 ppm to about 60 ppm, even more preferably from about 0 ppm to about 50 ppm, even more preferably from about 0 ppm to about 40 ppm, even more preferably from about 0 ppm to about 30 ppm, even more preferably from about 0 ppm to about 20 ppm, and most preferably from about 0 ppm to about 10 ppm.

E3. The surfactant of any of paragraphs A3-D3, wherein the ethoxylated fatty alcohol comprises 2-tridecyl alcohol ethoxylate, alcohol ethoxylate-2, 3-tridecyl alcohol, alcohol ethoxylate-1, alcohol ethoxylate 1.8, or a combination thereof.

F3. A surfactant comprises a sulfated ethoxylated alcohol with 100 ppm or less by weight of diethylene glycol monosulfate sodium.

G3. The surfactant of paragraph F3, wherein the amount of diethyelene glycol monosulfate sodium is from about 0 ppm to about 100 ppm, from about 0 ppm to about 90 ppm, from about 0 ppm to about 80 ppm, from about 0 ppm to about 70 ppm, from about 0 ppm to about 60 ppm, from about 0 ppm to about 50 ppm, from about 0 ppm to about 40 ppm, from about 0 ppm to about 30 ppm, from about 0 ppm to about 20 ppm, or from about 0 ppm to about 10 ppm.

H3. The surfactant of paragraph F3 or G3, wherein the sulfated ethoxylated alcohol comprises sodium laureth-1 sulfate, sodium laureth-1.8 sulfate, sodium laureth-2 sulfate, sodium laureth-2.5 sulfate, sodium laureth-3 sulfate, sodium trideceth-2 sulfate, sodium trideceth-3 sulfate, or a combination thereof.

I3. A cleansing composition comprises a surfactant as described in any of paragraphs A3-H3 and water.

J3. The cleansing composition of paragraph I3, wherein the water is present at a level of a 10% to about 90%; preferably from about 20% to about 80%, more preferably from about 30% to about 75%; or even more preferably from about 40% to about 75%.

K3. A method of slowing dioxane conversion, comprising: buffering a pH of an ethoxylated fatty alcohol raw material to about 6 to about 11, preferably from about 7 to about 10.5; more preferably from about 7 to about 8; even more preferably from about 9.5 to about 11, or most preferably from about 10 to about 11, wherein the pH is measured as a 10% solution.

L3. A method of slowing dioxane conversion, comprising: buffering a pH of a sulfated ethoxylated alcohol surfactant raw material to about 6 to about 11, preferably from about 7 to about 10.5; more preferably from about 7 to about 8; even more preferably from about 9.5 to about 11, or most preferably from about 10 to about 11, wherein the pH is measured as a 10% solution.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A surfactant comprising an ethoxylated fatty alcohol with 100 ppm or less by weight of free glycol or a sulfated ethoxylated alcohol with 100 ppm or less by weight of diethylene glycol monosulfate sodium.

2. The surfactant of claim 1, wherein the free glycol comprises an ethylene glycol oligomer.

3. The surfactant of any of claims 1 or 2, wherein the ethylene glycol oligomer comprises a chemical formula of HO(CH₂CH₂O)ₙH, wherein n is from 2 to about 30, preferably from 2 to about 25, more preferably from 2 to about 15, even more preferably from 2 to about 10, and most preferably from 2 to about 5.

4. The surfactant of any of claims 1 to 3 , wherein the amount of free glycol in the ethoxylated fatty alcohol is from about 0 ppm to about 100 ppm, preferably from about 0 ppm to about 90 ppm, more preferably from about 0 ppm to about 80 ppm, even more preferably from about 0 ppm to about 70 ppm, even more preferably from about 0 ppm to about 60 ppm, even more preferably from about 0 ppm to about 50 ppm, even more preferably from about 0 ppm to about 40 ppm, even more preferably from about 0 ppm to about 30 ppm, even more preferably from about 0 ppm to about 20 ppm, and most preferably from about 0 ppm to about 10 ppm.

5. The surfactant of any of claims 1 to 4 , wherein the ethoxylated fatty alcohol comprises 2-tridecyl alcohol ethoxylate, alcohol ethoxylate-2, 3-tridecyl alcohol, alcohol ethoxylate-1, alcohol ethoxylate 1.8, or a combination thereof.

6. The surfactant of claim 1, wherein the amount of diethyelene glycol monosulfate sodium is from about 0 ppm to about 100 ppm, from about 0 ppm to about 90 ppm, from about 0 ppm to about 80 ppm, from about 0 ppm to about 70 ppm, from about 0 ppm to about 60 ppm, from about 0 ppm to about 50 ppm, from about 0 ppm to about 40 ppm, from about 0 ppm to about 30 ppm, from about 0 ppm to about 20 ppm, or from about 0 ppm to about 10 ppm.

7. The surfactant of any of claims 1 or 6 , wherein the sulfated ethoxylated alcohol comprises sodium laureth-1 sulfate, sodium laureth-1.8 sulfate, sodium laureth-2 sulfate, sodium laureth-2.5 sulfate, sodium laureth-3 sulfate, sodium trideceth-2 sulfate, sodium trideceth-3 sulfate, or a combination thereof.

8. A cleansing composition comprising a surfactant as described in any of claims 1 to 7 and water.

9. The cleansing composition of claim 8, wherein the water is present at a level of a 10% to about 90%; preferably from about 20% to about 80%, more preferably from about 30% to about 75%; or even more preferably from about 40% to about 75%.

10. Use of a reduced glycol ethoxylated material to produce a reduced glycol sulfate ethoxylated sulfate surfactant.
